(19) United States
(12) Patent Application Publication
(10) Pub. No.: US 2007/0014842 A1
(43) Pub. Date: Jan. 18, 2007

(54) Pharmaceutical liposomal compositions

(76) Inventors: Denis Martin, St-Augustin-de-Desmaures (CA); Stephane Rioux, Ohain (BE)

Correspondence Address: MILLEN, WHITE, ZELANO & BRANIGAN, PC, 2200 CLARENDON BLVD, SUITE 1400, ARLINGTON, VA, 22201, US

(21) Appl. No.: 11/369,183

(22) Filed: Mar. 7, 2006

Related U.S. Application Data
(60) Provisional application No. 60/658,815, filed on Mar. 7, 2005.

(51) Int. Cl. A61K 39/095; A61K 9/127; C12Q 1/68; G01N 33/574

(52) U.S. Cl. 424/450; 424/249.1; 424/250.1; 435/7.23; 435/6

(57) ABSTRACT

Pharmaceutical compositions comprising a liposome associated to N. meningitidis polypeptides fragments or analogs thereof or corresponding DNA fragments, can be used to prevent, diagnose and/or treat neisserial infections.

This application claims the benefit of the filing date of U.S. Provisional Application Ser. No. 60/658,815 filed Mar. 7, 2005 which is incorporated by reference herein.

### FIELD OF THE INVENTION

The present invention is related to pharmaceutical compositions comprising a liposome associated to N. meningitidis polypeptides or corresponding DNA fragments, which may be used to prevent, diagnose and/or treat neisserial infections.

### BACKGROUND OF THE INVENTION

N. meningitidis is a major cause of death and morbidity throughout the world. N. meningitidis causes both endemic and epidemic diseases, principally meningitidis and meningococcemia [Tzeng, Y-L and D. S. Stephens, Microbes and Infection, 2, p. 687 (2000); Pollard, A. J. and C. Frasch, Vaccine, 19, p. 1327 (2001); Morley, S. L, and A. J. Pollard, Vaccine, 20, p. 666 (2002)]. It has been well documented that serum bactericidal activity is the major defence mechanism against N. meningitidis and that protection against invasion by bacteria correlates with the presence in the serum of anti-meningococcal antibodies [Goldschneider et al. J. Exp. Med. 129, p. 1307 (1969); Goldschneider et al. J. Exp. Med. 129, p. 1327 (1969)].

N. meningitidis are subdivided into serological groups according to the presence of capsular antigens. Currently, 12 serogroups are recognized, but serogroups A, B, C, Y, and W135 are most commonly found. Within serogroups, different serotypes, subtypes and immunotypes can be identified based on the outer membrane proteins and lipopolysaccharides [Frasch et al. Rev. Infect. Dis., 7, p. 504 (1985)].

The capsular polysaccharide vaccines presently available are not effective against all N. meningitidis isolates and do not effectively induce the production of protective antibodies in young infants [Tzeng, Y-L and D. S. Stephens, Microbes and Infection, 2, p. 687 (2000); Pollard, A. J. and C. Frasch, Vaccine, 19, p. 1327 (2001); Morley, S. L, and A. J. Pollard; Vaccine, 20, p. 666 (2002)]. The capsular polysaccharides of serogroups A, C, Y, and W135 are presently used in vaccines against this organism. These polysaccharide vaccines are effective in the short term, however vaccinated subjects do not develop an immunological memory, so they must be revaccinated within a three-year period to maintain their level of resistance.

Furthermore, these vaccines do not induce sufficient levels of bactericidal antibodies to obtain the desired protection in very young children, who are the principal victims of this disease. No effective vaccine against serogroup B isolates is presently available even though these organisms are one of the primary causes of meningococcal diseases in developed countries. Furthermore, the presence of closely similar, cross-reactive structures in the glycoproteins of neonatal human brain tissue might discourage attempts at improving the immunogenicity of serogroup B polysaccharide [Finne et al. Lancet, p. 355 (1983)].

To obtain a more effective vaccine, other N. meningitidis surface antigens such as lipopolysaccharide, pili, proteins are under investigation. The presence of human immune response and bactericidal antibodies against certain of these proteinaceous surface antigens in the sera of immunized volunteers and convalescent patients was demonstrated [Mandrell and Zollinger, Infect. Immun, 57, p. 1590 (1989); Poolman et al. Infect. Immun., 40, p. 398 (1983); Rosenquist et al. J. Clin. Microbiol., 26, p. 1543 (1988); Wedege and Froholm Infect. Immun. 51, p. 571 (1986); Wedege and Michaelsen, J. Clin Microbiol., 25, p. 1349 (1987)].

One of the main problems with most of the already described meningococcal surface proteins is their antigenic heterogeneity. Indeed, the interstrain variability of the major outer membrane proteins restricts their protective efficacy to a limited number of antigenically related meningococcal strains. Several strategies based on either outer membrane vesicles, which contained most of the major surface proteins, or purified outer membrane proteins, are presently being explored in order to broaden the protective potential of protein-based meningococcal vaccines [Tzeng, Y-L and D. S. Stephens, Microbes and Infection, 2, p. 687 (2000); Pollard, A. J. and C. Frasch, Vaccine, 19, p. 1327 (2001); Morley, S. L, and A. J. Pollard, Vaccine, 20, p. 666 (2002)]. The identification of universal or, at least widely, distributed proteins with antigenically conserved surface-exposed regions would offer a solution to the great heterogeneity of the major meningococcal outer membrane proteins. One such an antigen, named NspA for Neisserial surface protein A, was disclosed in PCT/WO/96/29412 and is herein incoporated by reference.

Monoclonal antibodies (Mabs) directed against the NspA protein reacted with more than 99% of the meningococcal strains tested, clearly indicating that highly conserved antigenic regions were present on this protein [Martin et al. J. Exp. Med., 185, p. 1173 (1997); Cadieux et al. Infect. Immun., 67, p. 4955, (1999)]. Immunoelectron microscopy and flow cytofluorometric data clearly demonstrated that the NspA protein is present at the surface of intact meningococcal cells and that this protein is evenly distributed at the cell surface [Cadieux et al. Infect. Immun., 67, p. 4955, (1999)]. The gene encoding for this protein was cloned and sequenced [Martin et al. J. Exp. Med., 185, p. 1173 (1997)]. Comparison of this sequence with the sequences compiled in the available databases indicated that the nspA gene shared homologies with members of the Neisserial opacity protein family (Opa), which are also found in the meningococcal outer membrane. DNA hybridization clearly established that the nspA gene is present in the genome of all meningococcal strains tested, but it also indicated that highly conserved homologs were also present in the closely related species N. gonorrhoeae, N. lactamica and N. polysaccharea. Characterization of the gonococcal NspA protein was presented previously [Plante et al. Infect. Immun., 67, p. 2855 (1999)]. The conclusive proof about the high level of molecular conservation (>96% identity) of this protein was obtained following the cloning and sequencing of additional nspA genes from divergent serogroups A, B and C meningococcal strains [Martin et al. J. Exp. Med., 185, p. 1173 (1997); Cadieux et al. Infect. Immun. 67, p. 4955, (1999); Moe et al., Infect. Immun., 67, p. 2855 (1999)]. The nspA gene was cloned into the expression vector pWKS30 in order to obtain sufficient amount of purified protein to evaluate its protective potential in a mouse model of infection [Martin et al. J. Exp. Med., 185, p. 1173 (1997)]. BALB/c mice were immunized three times with 20 μg of immunoaffinity-purified recombinant NspA protein and the mice were then challenged with a lethal dose of a serogroup B strain. 80% of the NspA-immunized mice survived the bacterial challenge comparatively to less than 20% in the control groups. Analysis of the sera collected from the mice that survived the lethal meningococcal challenge revealed the presence of cross-reactive antibodies, which attached to and killed the four serogroup B strains tested. In addition, passive immunization of mice with NspA-specific MAbs confirmed the protective potential of the protein. Indeed, administration of an NspA-specific MAb 18 h before challenge reduced by more than 75% the levels of bacteremia recorded for mice challenged with 10 out of 11 meningococcal strains tested [Cadieux et al. Infect. Immun., 67, p. 4955, (1999)]. These results indicated that this highly conserved protein can induce protective immunity against meningococcal infection.

Studies with recombinant meningococcal surface-exposed PorA, PorB and Opc proteins have indicated that the efficient production of bactericidal antibodies was often dependent on the refolding of the recombinant protein to generate the native conformation [Christodoulides et al. Microbiol., 144, p. 3027, (1998); Idanpaan-Heikkila et al. Vaccine, 13, p. 1501-(1995); Muttilainen et al., Microb. Pathog., 18, p. 365 (1995); Muttilainen et al., Microb. Pathog., 18, p. 423 (1995); Ward et al. Microb. Pathog., 21, p. 499, (1996); Wright et al. Infect. Immun., 70, p. 4028 (2002); Musacchio et al., Vaccine, 15, p. 751 (1996)]. One method used to favour the refolding of recombinant surface proteins is their incorporation into liposomes.

However, there remains an unmet need for pharmaceutical compositions that may be used for the prophylaxis, diagnosis and/or therapy of neisserial infections.

### SUMMARY OF THE INVENTION

According to one aspect, the present invention relates to a pharmaceutical composition comprising a liposome associated with polypeptides comprising SEQ ID No: 2 or fragments or analogs thereof.

In other aspects, there are provided processes for producing pharmaceutical compositions of the invention, methods for delivering pharmaceutical compositions of the invention to the host, method of uses of pharmaceutical compositions of the invention.

### BRIEF DESCRIPTION OF THE DRAWINGS

FIG. 1 represents the nucleotide (SEQ ID No:1) and amino acid (SEQ ID No.:2) sequences of the gene encoding the N. meningitidis strain 608B NspA protein.

FIG. 2 represents the 3-D model of the meningococcal NspA protein. This model was developed from the crystal structure of the refolded E. coli OmpA (PDB: 1QJP) [Pautsch, A. and GE Schulz, J. Mol. Biol., 298, p. 273 (2000)] using Swiss-Pdb Viewer [Guex, N. and MC Peitsch, Electrophoresis, 18, p. 2714 (1997)]. The eight transmembrane β-strands are connected with three tight turns (T) on the periplasmic side and four surface-exposed loops (L1, L2, L3, L4) on the outer surface of the bacteria. The amino acid residues, which interact with the membrane interphase are represented as balls and sticks. This figure was prepared using 3D-Mol Viewer from vector NTI suite 7.0 (InforMax, Inc.).

FIG. 3 represents the evaluation by flow cytometry of the accessibility of NspA-specific MAbs at the surface of two serogroup B meningococcal strain 608B (B:2a:P1.2:L3), CU385 (B:4:P1.15:L3,7,9), one serogroup A strain F8238 (A:4,21) and one serogroup C strain C11 (NT:P1.1:L3,7,9). Exponentially growing meningococcal cells were sequentially incubated with NspA-specific or control MAbs, followed by FITC-conjugated anti-mouse immunoglobulin secondary antibody. The bactericidal activity of each MAb is presented as the concentration of antibody resulting in a 50% decrease of CFU per mL after 60 min of incubation compared to control CFU: ++, between 0.5-49 μg of antibody/mL; +, between 50-99 μg of antibody/mL; − no bactericidal activity at >100 μg of antibody/mL.

FIG. 4 depicts the evaluation of the binding of polyclonal anti-NspA rabbit antisera to Neisseria meningitidis strains 608B (B:2a:P1.2), BZ198 (B:NT:P−), S3446 (B:14:P1.23,14) and H355 (B:15:P1.15), as determined by indirect fluorescence flow cytometry. Rabbits were immunized with 100 μg of rNspA incorporated into different liposome formulations. Exponentially growing meningococcal cells were sequentially incubated with pre-bleed or hyperimmune sera, followed by fluorescein isothiocyanate (FITC)-conjugated anti-rabbit immunoglobulin secondary antibody. All sera were tested at a dilution of 1:20. In each graph, the left peak-represents the binding of pre-bleed rabbit serum, while the right peak represents the binding of the corresponding hyperimmune serum against intact meningococcal cells.

### DETAILED DESCRIPTION OF THE INVENTION

The present invention provides pharmaceutical compositions comprising a liposome associated with N. meningitidis polypeptides which may be used to prevent, diagnose and/or treat Neisserial infections.

According to one aspect, the present invention relates to pharmaceutical composition comprising a liposome associated with polypeptides comprising SEQ ID No: 2 or fragments or analogs thereof.

According to one aspect, the present invention relates to pharmaceutical composition comprising a liposome associated with polypeptides comprising SEQ ID No: 2.

According to one aspect, the present invention relates to pharmaceutical composition comprising a liposome associated with polypeptides consisting of SEQ ID No: 2 or fragments or analogs thereof.

According to one aspect, the present invention relates to pharmaceutical composition comprising a liposome associated with polypeptides consisting of SEQ ID No: 2.

According to one aspect, the present invention relates to pharmaceutical composition comprising a liposome associated with epitope bearing portions of a polypeptide comprising SEQ ID No: 2 or fragments or analogs thereof.

According to one aspect, the present invention relates to pharmaceutical composition comprising a liposome associated with epitope bearing portions of a polypeptide comprising SEQ ID No 2.

According to one aspect, the present invention provides a pharmaceutical composition comprising a liposome associated with an isolated polypeptide chosen from:
(a) a polypeptide having at least 70% identity to a second polypeptide comprising SEQ ID No: 2 or fragments or analogs thereof;
(b) a polypeptide having at least 80% identity to a second polypeptide comprising SEQ ID No: 2 or fragments or analogs thereof;
(c) a polypeptide having at least 95% identity to a second polypeptide comprising SEQ ID No: 2 or fragments or analogs thereof;
(d) a polypeptide comprising SEQ ID No: 2 or fragments or analogs thereof;
(e) a polypeptide capable of raising antibodies having binding specificity for a polypeptide comprising SEQ ID No: 2 or fragments or analogs thereof;
(f) an epitope bearing portion of a polypeptide comprising SEQ ID No: 2 or fragments or analogs thereof;
(g) the polypeptide of (a), (b), (c), (d), (e) or (f) wherein the N-terminal Met residue is deleted;
(h) the polypeptide of (a), (b), (c), (d), (e), (f) or (g) wherein the secretory amino acid sequence is deleted.

According to one aspect, the present invention provides a pharmaceutical composition comprising a liposome associated with an isolated polypeptide chosen from:
(a) a polypeptide having at least 70% identity to a second polypeptide comprising SEQ ID No: 2;
(b) a polypeptide having at least 80% identity to a second polypeptide comprising SEQ ID No: 2;
(c) a polypeptide having at least 95% identity to a second polypeptide comprising SEQ ID No: 2;
(d) a polypeptide comprising SEQ ID No: 2;
(e) a polypeptide capable of raising antibodies having binding specificity for a polypeptide comprising SEQ ID No: 2;
(f) an epitope bearing portion of a polypeptide comprising SEQ ID No: 2;
(g) the polypeptide of (a), (b), (c), (d), (e) or (f) wherein the N-terminal Met residue is deleted;
(h) the polypeptide of (a), (b), (c), (d), (e), (f) or (g) wherein the secretory amino acid sequence is deleted.

According to one aspect, the present invention provides a pharmaceutical composition comprising a liposome associated with an isolated polynucleotide chosen from:
(a) a polynucleotide encoding a polypeptide having at least 70% identity to a second polypeptide comprising SEQ ID No: 2 or fragments or analogs thereof;
(b) a polynucleotide encoding a polypeptide having at least 80% identity to a second polypeptide comprising SEQ ID No: 2 or fragments or analogs thereof;
(c) a polynucleotide encoding a polypeptide having at least 95% identity to a second polypeptide comprising SEQ ID No: 2 or fragments or analogs thereof;
(d) a polynucleotide encoding a polypeptide comprising SEQ ID No: 2 or fragments or analogs thereof;
(e) a polynucleotide encoding a polypeptide capable of raising antibodies having binding specificity for a polypeptide comprising SEQ ID No: 2 or fragments or analogs thereof;
(f) a polynucleotide encoding an epitope bearing portion of a polypeptide comprising SEQ ID No: 2 or fragments or analogs thereof;
(g) a polynucleotide comprising SEQ ID No: 1 or fragments or analogs thereof;
(h) a polynucleotide that is complementary to a polynucleotide in (a), (b), (c), (d), (e), (f) or (g)

According to one aspect, the present invention provides pharmaceutical composition comprising a liposome associated with an isolated polynucleotide comprising a polynucleotide chosen from:
(a) a polynucleotide encoding a polypeptide having at least 70% identity to a second polypeptide comprising SEQ ID No: 2;
(b) a polynucleotide encoding a polypeptide having at least 80% identity to a second polypeptide comprising SEQ ID No: 2;
(c) a polynucleotide encoding a polypeptide having at least 95% identity to a second polypeptide comprising SEQ ID No: 2;
(d) a polynucleotide encoding a polypeptide comprising SEQ ID No: 2;
(e) a polynucleotide encoding a polypeptide capable of raising antibodies having binding specificity for a polypeptide comprising SEQ ID No: 2;
(f) a polynucleotide encoding an epitope bearing portion of a polypeptide comprising SEQ ID No: 2;
(g) a polynucleotide comprising SEQ ID No: 1;
(h) a polynucleotide that is complementary to a polynucleotide in (a), (b), (c), (d), (e), (f) or (g).

Those skilled in the art will appreciate that the invention includes a pharmaceutical composition comprising a liposome and DNA molecules, i.e. polynucleotides and their complementary sequences that encode analogs such as mutants, variants, homologues and derivatives of such polypeptides, as described herein in the present patent application. The invention also includes RNA molecules corresponding to the DNA molecules of the invention. In addition to the DNA and RNA molecules, the invention includes the corresponding polypeptides and monospecific antibodies that specifically bind to such polypeptides.

As used herein, “associated with” means that the polypeptides of the invention are at least partially embedded in the liposome membrane, and preferably are not covalently linked to the lipids. The polypeptides may also be bonded to a lipid fatty acid “tail” which itself is embedded in the membrane.

In a further embodiment, the pharmaceutical compositions comprising a liposome associated with polypeptides in accordance with the present invention are antigenic.

In a further embodiment, the pharmaceutical compositions comprising a liposome associated with polypeptides in accordance with the present invention are immunogenic.

In a further embodiment, the pharmaceutical compositions comprising a liposome associated with polypeptides in accordance with the present invention can elicit an immune response in a host.

In a further embodiment, the present invention also relates to pharmaceutical compositions comprising a liposome associated with polypeptides which are able to raise antibodies having binding specificity to the polypeptides of the present invention as defined above.

An antibody that “has binding specificity” is an antibody that recognizes and binds the selected polypeptide but which does not substantially recognize and bind other molecules in a sample, e.g., a biological sample, which naturally includes the selected peptide. Specific binding can be measured using an ELISA assay in which the selected polypeptide is used as an antigen.

In accordance with the present invention, “protection” in the biological studies is defined by a significant increase in the production of bacterial antibodies or a significant increase in the bactericidal activity

In an additional aspect of the invention there are provided pharmaceutical compositions comprising a liposome associated with immunogenic and/or antigenic fragments of the polypeptides of the invention, or of analogs thereof.

The fragments of the present invention should include one or more such epitopic regions or be sufficiently similar to such regions to retain their immunogenic and/or antigenic properties.

Thus, for fragments according to the present invention the degree of identity is perhaps irrelevant, since they may be 100% identical to a particular part of a polypeptide or analog thereof as described herein. The present invention further provides an immunogenic fragment of a polypeptide of the invention, said fragment being a contiguous portion of the polypeptide of the invention. The present invention further provides fragments having at least 10 contiguous amino acid residues from the polypeptide sequences of the present invention. In one embodiment, at least 15 contiguous amino acid residues. In one embodiment, at least 20 contiguous amino acid residues. In one embodiment, at least 30 contiguous amino acid residues. In one embodiment, at least 40 contiguous amino acid residues. In one embodiment, at least 50 contiguous amino acid residues. In one embodiment, at least 100 contiguous amino acid residues. In one embodiment, at least 150 contiguous amino acid residues.

The present invention further provides a fragment which has the same or substantially the same immunogenic activity as the polypeptide comprising Seq. ID no. 2. The fragment (when coupled to a carrier, if necessary) is capable of raising an immune response which recognizes the NspA polypeptide.

Such an immunogenic fragment may include, for example, the NspA polypeptide lacking an N-terminal leader peptide, and/or a transmembrane domain and/or external loops and/or turns. The present invention further provides a fragment of NspA comprising substantially all of the extra cellular domain of a polypeptide which has at least 70% identify, preferably 80% identity, more preferably 95% identity, to a second polypeptide comprising Seq. ID No. 2, over the entire length of said sequence.

The present invention further provides pharmaceutical compositions comprising a liposome associated with fragments which comprise a B-cell or T-helper epitope.

The present invention further provides pharmaceutical compositions comprising a liposome associated with fragment that may be part of a larger polypeptide. It can be advantageous to include an additional amino acid sequence which contains secretory or leader sequences, or sequences which aid in purification such as multiple histidine residues, or an additional sequence which increases stability during recombinant production, or an additional polypeptide or lipid tail sequences which increase the immunogenic potential of the final polypeptide.

The skilled person will appreciate that pharmaceutical compositions comprising a liposome associated with analogs of the polypeptides of the invention will also find use in the context of the present invention, i.e. as antigenic/immunogenic material. Thus, for instance proteins or polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 80% identity with those sequences illustrated in the figures or fragments thereof. That is, 80% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

These substitutions are those having a minimal influence on the secondary structure and hydropathic nature of the polypeptide. Preferred substitutions are those known in the art as conserved, i.e. the substituted residues share physical or chemical properties such as hydrophobicity, size, charge or functional groups. These include substitutions such as those described by Dayhoff, M. in Atlas of Protein Sequence and Structure 5, 1978 and by Argos, P. in EMBO J. 8, 779-785, 1989. For example, amino acids, either natural or unnatural, belonging to one of the following groups represent conservative changes:

ala, pro, gly, gln, asn, ser, thr, val;

cys, ser, tyr, thr;

val, ile, leu, met, ala, phe;

lys, arg, orn, his;

and phe, tyr, trp, his.

The preferred substitutions also include substitutions of D-enantiomers for the corresponding L-amino acids.

The percentage of homology is defined as the sum of the percentage of identity plus the percentage of similarity or conservation of amino acid type.

In one embodiment, analogs of polypeptides of the invention will have about 70% identity with those sequences illustrated in the figures or fragments thereof. That is, 70% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

In one embodiment, analogs of polypeptides of the invention will have about 70% homology with those sequences illustrated in the figures or fragments thereof. In a further embodiment, polypeptides will have greater than 80% homology. In a further embodiment, polypeptides will have greater than 85% homology. In a further embodiment, polypeptides will have greater than 90% homology. In a further embodiment, polypeptides will have greater than 95% homology. In a further embodiment, polypeptides will have greater than 99% homology. In a further embodiment, analogs of polypeptides of the invention will have fewer than about 20 amino acid residue substitutions, modifications or deletions and more preferably less than 10.

One can use a program such as the CLUSTAL program to compare amino acid sequences. This program compares amino acid sequences and finds the optimal alignment by inserting spaces in either sequence as appropriate. It is possible to calculate amino acid identity or homology for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

It is well known that it is possible to screen an antigenic polypeptide to identify epitopic regions, i.e. those regions which are responsible for the polypeptide's antigenicity or immunogenicity. Methods for carrying out such screening are well known in the art. Thus, the fragments of the present invention should include one or more such epitopic regions or be sufficiently similar to such regions to retain their antigenic/immunogenic properties.

Thus, what is important for analogs, derivatives and fragments is that they possess at least a degree of the antigenicity/immunogenicity of the protein or polypeptide from which they are derived.

Furthermore, in those situations where amino acid regions are found to be polymorphic, it may be desirable to vary one or more particular amino acids to more effectively mimic the different epitopes of the different N. meningitidis strains.

In a further embodiment, the present invention also relates to pharmaceutical compositions comprising a liposome associated with chimeric polypeptides which comprise one or more polypeptides or fragments or analogs thereof of the invention.

In a further embodiment, the present invention also relates to pharmaceutical compositions comprising a liposome associated with chimeric polypeptides comprising two or more polypeptides comprising SEQ ID No: 2 or fragments or analogs thereof; provided that the polypeptides are linked as to formed a chimeric polypeptide.

In a further embodiment, the present invention also relates to pharmaceutical compositions comprising a liposome associated with chimeric polypeptides comprising two or more polypeptides comprising SEQ ID No: 2 provided that the polypeptides are linked as to form a chimeric polypeptide.

Preferably, a fragment, analog or derivative of a polypeptide of the pharmaceutical compositions of the invention will comprise at least one antigenic region i.e. at least one epitope.

In a particular embodiment, polypeptide fragments and analogs comprised in the pharmaceutical compositions of the invention do not contain a starting residue, such as methionine (Met) or valine (Val). Preferably, polypeptides will not incorporate a leader or secretory sequence (signal sequence). The signal portion of a polypeptide of the invention may be determined according to established molecular biological techniques. In general, the polypeptide of interest may be isolated from a N. meningitidis culture and subsequently sequenced to determine the initial residue of the mature protein and therefore the sequence of the mature polypeptide.

It is understood that polypeptides for the pharmaceutical compositions of the invention can be produced and/or used without their start codon (methionine or valine) and/or without their leader peptide to favor production and purification of recombinant polypeptides. It is known that cloning genes without sequences encoding leader peptides will restrict the polypeptides to the cytoplasm of E. coli and will facilitate their recovery (Glick, B. R. and Pasternak, J. J. (1998) Manipulation of gene expression in prokaryotes. In “Molecular biotechnology: Principles and applications of recombinant DNA”, 2nd edition, ASM Press, Washington D.C., p. 109-143).

The NspA protein was shown to be antigenically highly conserved and present in the outer membrane of N. meningitidis where it is accessible to specific antibodies.

In vitro folding of the NspA may improve the production of bactericidal antibodies. One of the methods that can be used to improve folding of this membrane protein is its incorporation into a liposome.

Liposomes are made of phospholipids and other polar amphiles, which form closed concentric bilayer membranes [summarized in Gregoriades, G., Immunology Today, 11, 3, 89 (1990); Lasic, D., American Scientist, 80, p. 20 (1992); Remington's on Pharmaceutical Sciences, 18th ed., 1990, Mack Publishing Co., Pennsylvania., p. 1691]. The primary constituent of liposomes are lipids, which have a polar hydrophilic “head” attached to a long, nonpolar, hydrophobic “tail”. The hydrophilic head typically consists of a phosphate group, while the hydrophobic tail is made of two long hydrocarbon chains. Since the lipid molecules have one part that is water-soluble and another part that is not, they tend to aggregate in ordered structures that sequester the hydrophobic tails from water molecules. In the process, liposomes can entrap water and solutes in their interior, or molecules with hydrophobic regions can also be incorporated directly into the liposomal membranes. Many phospholipids, alone or in combination, with other lipids will form liposomes. By convention, liposomes are categorized by size, and a 3-letter acronym is used to designate the type of liposome being discussed. Multilamellar vesicles are designated “MLV”, large unilamellar vesicles “LUV”, small unilamellar vesicles “SUV”. These designations are sometimes followed by the chemical composition of the liposome. Nomenclature and a summary of known liposomes is described in Storm et al, 1998, PSIT, 1:19-31. Liposomes are efficient in hleping membrane proteins refolding and are also efficient adjuvant boosting the humoral as well as the cellular immune response against an antigen.

The invention provides pharmaceutical compositions comprising liposomes having a protein to lipid ratio between about 1 to 50 to about 1 to 1500.

The invention provides pharmaceutical compositions comprising liposomes constituted from phospholipids. These phospholipids can be synthetized or extracted from bacterial cells, soybean, eggs.

The invention provides a process for the incorporation of recombinant NspA polypeptides into different liposome formulations.

Liposomes can be prepared with various synthetic phospholipids (List 1) or bacterial phospholipids and/or cholesterol, which can be combined at different ratios.

The invention provides a method for extracting lipids from bacterial cells in order to generate liposome formulations from bacterial origin. Complex lipid mixtures can be extracted from several bacterial species. These species could include but are not limited to: Neisseria spp, Haemophilus spp, Pseudomonas spp, Bacteriodes spp, Legionella spp, Vibrio spp, Brucella spp,. Bordetella spp, Campylobacter spp, Klebsiella spp, Salmonella spp, Shigella spp, Proteus spp, and Yersinia spp. Other species can be found in Bergey's Manual of Determinative Bacteriology (1974) (Baltimore). In a prefered embodiment, complex lipid mixtures are extracted from E. coli, N. meningitidis, or N. lactamica.

The liposomes of the invention can be prepared from a variety of vesicle-forming lipids including phosphatidyl ethers and esters, such as phosphatidylethanloamine (PE), phosphatidylserine (PS), phosphatidylglycerol (PG) and phosphatidylcholine (PC) but also from glycerides, such as dioleoylglycerosuccinate; cerebrosides; gangliosides, sphyngomyelin; steroids, such as cholesterol; and other lipids, as well as excipients such as Vitamin E or Vitamin C palmitate.

List 1 provides a partial list of synthetic lipids that can be used to prepare NspA-liposome preparations. Other lipids can be used and are described in Remington's on Pharmaceutical Sciences, 18th ed., 1990, Mack Publishing Co., Pennsylvania, p. 390.

List 1. List of synthetic lipids used to prepare NspA-liposome preparations.
1,2-Dilauroyl-sn-Glycero-3-Phosphate (DLPA),
Dimyristoyl-sn-Glycero-3-Phosphate (DMPA),
1,2-Dipalmitoyl-sn-Glycero-3-Phosphate (DPPA),
1,2-Distearoyl-sn-Glycero-3-Phosphate (DSPA),
1,2-Dioleoyl-sn-Glycero-3-Phosphate (DOPA),
1-Palmitoyl-2-Oleoyl-sn-Glycero-3-Phosphate (POPA),
1,2-Dilauroyl-sn-Glycero-3-Phosphocholine (DLPC),
1,2-Ditridecanoyl-sn-Glycero-3-Phosphocholine,
1,2-Dimyristoyl-sn-Glycero-3-Phosphocholine (DMPC),
1,2-Dipentadecanoyl-sn-Glycero-3-Phosphocholine,
1,2-Dipalmitoyl-sn-Glycero-3-Phosphocholine (DPPC),
1,2-Diheptadecanoyl-sn-Glycero-3-Phosphocholine,
1,2-Distearoyl-sn-Glycero-3-Phosphocholine (DSPC),
1,2-Dimyristoleoyl-sn-Glycero-3-Phosphocholine,
1,2-Dipalmitoleoyl-sn-Glycero-3-Phosphocholine,
1,2-Dioleoyl-sn-Glycero-3-Phosphocholine (DOPC),
1-Myristoyl-2-Palmitoyl-sn-Glycero-3-Phosphocholine,
1-Myristoyl-2-Stearoyl-sn-Glycero-3-Phosphocholine,
1-Palmitoyl-2-Myristoyl-sn-Glycero-3-Phosphocholine,
1-Palmitoyl-2-Stearoyl-sn-Glycero-3-Phosphocholine,
1-Palmitoyl-2-Oleoyl-sn-Glycero-3-Phosphocholine (POPC),
1-Palmitoyl-2-Linoleoyl-sn-Glycero-3-Phosphocholine,
1,2-Dilauroyl-sn-Glycero-3-Phosphoethanolamine (DLPE); 1,2-Dimyristoyl-sn-Glycero-3-Phosphoethanolamine (DMPE),
1,2-Dipalmitoyl-sn-Glycero-3-Phosphoethanolamine (DPPE),
1,2-Dipalmitoleoyl-sn-Glycero-3-Phosphoethanolamine,
1,2-Distearoyl-sn-Glycero-3-Phosphoethanolamine (DSPE),
1,2-Dioleoyl-sn-Glycero-3-Phosphoethanolamine (DOPE),
1-Palmitoyl-2-Oleoyl-sn-Glycero-3-Phosphoethanolamine (POPE),
1,2-Dilaurloyl-sn-Glycero-3-[Phospho-RAC-(1-glycerol)] (DLPG),
1,2-Dimyristoyl-sn-Glycero-3-[Phospho-RAC-(1-glycerol)] (DMPG),
1,2-Dipalmitoyl-sn-Glycero-3-[Phospho-RAC-(1-glycerol)] (DPPG),
1,2-Distearoyl-sn-Glycero-3-[Phospho-RAC-(1-glycerol)] (DSPG),
1,2-Dioleoyl-sn-Glycero-3-[Phospho-RAC-(1-glycerol)] (DOPG),
1-Palmitoyl-2-Oleoyl-sn-Glycero-3-[Phospho-RAC-(1-glycerol)] (POPG),
1,2-Dilauroyl-sn-Glycero-3-[Phospho-L-Serine] (DLPS),
1,2-Dimyristoyl-sn-Glycero-3-[Phospho-L-Serine] (DMPS),
1,2-Dipalmitoyl-sn-Glycero-3-[Phospho-L-Serine] (DPPS),
1,2-Distearoyl-sn-Glycero-3-[Phospho-L-Serine] (DSPS),
1,2-Dioleoyl-sn-Glycero-3-[Phospho-L-Serine] (DOPS),
1-Palmitoyl-2-Oleoyl-sn-Glycero-3-[Phospho-L-Serine] (POPS).

In a prefered embodiment, lipids are chosen from
1,2-Dimyristoyl-sn-Glycero-3-Phosphocholine (DMPC),
1,2-Dimyristoyl-sn-Glycero-3-[Phospho-L-Serine] (DMPS), and
1,2-Dimyristoyl-3-Trimethylammonium-Propane (DMTAP).

The fluidity and stability of the liposomal membrane will depend on the transition temperature (temperature at which hydrocarbon regions change from a quasicrystalline to a more fluid state) of the phospholipids.

Modifications of membrane fluidity, number of lamellae, vesicle size, surface charge, lipid to antigen ratio and localization of the antigen within the liposome can modulate the ajduvanticity of liposomal preparations.

The preparation of liposomes can be made by a number of different techniques including ethanol injection; ether infusion; detergent removal; solvent evaporation; evaporation of organic solvents from chloroform in water emulsions; extrusion of multilamellar vesicles through a nucleopore polycarbonate membrane; freezing and thawing of phospholipid mixtures, as well as sonication and homogenization.

Lipids can be dissolved in a suitable organic solvent or mixture of organic solvents, such as a chloroform:methanol solution in a round bottom glass flask and dried using a rotatory evaporator to achieve an even film on the vessel.

A protein-detergent solution containing the NspA protein and SDS can then be added to the lipid film and mixed gently until the film is dissolved. The solution is then dialysed against PBS buffer to remove detergent and to induce liposome formation.

Gel filtration can be used as an alternate method to induce the formation of NspA liposome from the NspA-OG-SDS-lipids mixed micellar solution and to remove detergents.

Some liposome formulations can also be prepared with an adjuvant such as lipophilic molecules such as Lipid A, monophosphoryl lipid A (MPLA), lipopolysaccharides such as QuilA, QS21, alum, MF59, p3CSS, MTP-PE, as well as water-soluble molecules, including cytokines such as interferons. In a preferred embodiment, the liposome composition comprises about 1-10% adjuvant(s). In a more preferred embodiment, the adjuvant is present in less than about 5%. The values may be vol/vol or wt/wt depending upon the adjuvant.

According to the present invention, the liposome plays a critical role in antigen delivery as the polypeptide-liposome composition is directly presented to the immune system following removal from the circulation by cells of the immune system. In addition, the choice of the immunostimulatory pathways can be altered by making changes to the lipid composition of the liposome. For example, different immunostimulatory molecules, such as Lipid A, muramyl di- and tripeptide-PE and cationic lipids can be formulated into the liposome.

In addition to helping membrane proteins refolding, liposomes are also efficient adjuvant boosting the humoral as well as the cellular immune response against an antigen. Modifications of membrane fluidity, number of lamellae, vesicle size, surface charge, lipid to antigen ratio and localization of the antigen within the liposome can modulate the adjuvanticity of liposomal preparations.

In a prefered embodiment, the lipid formulation contain between 0 and 25 mol % cholesterol.

According to another aspect of the invention, there are also provided (i) a composition of matter containing a polypeptide of the invention, together with a liposome, carrier, diluent or adjuvant; (ii) a pharmaceutical composition comprising a polypeptide of the invention and a liposome, carrier, diluent or adjuvant; (iii) a vaccine comprising a polypeptide of the invention and a liposome, carrier, diluent or adjuvant; (iv) a method for inducing an immune response against N. meningitidis, in a host, by administering to the host, an immunogenically effective amount of a pharmaceutical composition of the invention to elicit an immune response, e.g., a protective immune response to N. meningitidis; and particularly, (v) a method for preventing and/or treating a N. meningitidis infection, by administering a prophylactic or therapeutic amount of a pharmaceutical composition of the invention to a host in need.

According to another aspect of the invention, there are also provided (i) a composition of matter containing a polynucleotide of the invention, together with a liposome, carrier, diluent or adjuvant; (ii) a pharmaceutical composition comprising a polynucleotide of the invention and a liposome, carrier, diluent or adjuvant; (iii) a method for inducing an immune response against N. meningitidis, in a host, by administering to the host, an immunogenically effective amount of a pharmaceutical composition of the invention to elicit an immune response, e.g., a protective immune response to N. meningitidis; and particularly, (iv) a method for preventing and/or treating a N. meningitidis infection, by administering a prophylactic or therapeutic amount of a pharmaceutical composition of the invention to a host in need.

According to another aspect, there are provided pharmaceutical compositions comprising a liposome, one or more N. meningitidis polypeptides of the invention in a mixture with a pharmaceutically acceptable adjuvant. Suitable adjuvants include (1) oil-in-water emulsion formulations such as MF59™, SAF™, Ribi™; (2) Freund's complete or incomplete adjuvant; (3) salts i.e. AlK(SO₄)₂, AlNa(SO₄)₂, AlNH₄(SO₄)₂, Al(OH)₃, AlPO₄, silica, kaolin; (4) saponin derivatives such as Stimulon™ or particles generated therefrom such as ISCOMs (immunostimulating complexes); (5) cytokines such as interleukins, interferons, macrophage colony stimulating factor (M-CSF), tumor necrosis factor (TNF); (6) other substances such as carbon polynucleotides i.e. poly IC and poly AU, detoxified cholera toxin (CTB) and E. coli heat labile toxin for induction of mucosal immunity. A more detailed description of adjuvants is available in a review by M. Z. I Khan et al. in Pharmaceutical Research, vol. 11, No. 1 (1994) pp2-11, and also in another review by Gupta et al., in Vaccine, Vol. 13, No. 14, pp 1263-1276 (1995) and in WO 99/24578. Preferred adjuvants include QuilA™, QS21™, Alhydrogel™ and Adjuphos™.

Pharmaceutical compositions of the invention may be administered parenterally by injection, rapid infusion, nasopharyngeal absorption, dermoabsorption, or buccal or oral.

The term pharmaceutical composition is also meant to include antibodies. In accordance with the present invention, there is also provided the use of one or more antibodies having binding specificity for the polypeptides of the present invention for the treatment or prophylaxis of N. meningitidis infection and/or diseases and symptoms mediated by N. meningitidis infection.

Pharmaceutical compositions of the invention are used for the prophylaxis of neisserial infections and/or diseases and symptoms mediated by neisserial infections as described in Manual of Clinical Microbiology, P. R. Murray (Ed, in chief), E. J. Baron, M. A. Pfaller, F. C. Tenover and R. H. Yolken. ASM Press, Washington, D.C. seventh edition, 1999, 1773p.

In one embodiment, pharmaceutical compositions of the present invention are used for the treatment or prophylaxis of endemic and epidemic diseases, such as meningitidis and meningoccemia. In one embodiment, vaccine compositions of the invention are used for the treatment or prophylaxis of neisserial infections and/or diseases and symptoms mediated by neisserial infections. In a further embodiment; the neisserial infection is N. meningitidis, N. gonorrhoeae, N. lactamica or N. polysaccharea.

In a further embodiment, the invention provides a method for prophylaxis or treatment of N. meningitidis infection in a host susceptible to N. meningitidis infection comprising administering to said host a prophylactic or therapeutic amount of a composition of the invention.

As used in the present application, the term “host” includes mammals. In a further embodiment, the mammal is human.

In a particular embodiment, pharmaceutical compositions are administered to those hosts at risk of N. meningitidis infection such as neonates, infants, children, elderly and immunocompromised hosts.

In a particular embodiment, pharmaceutical compositions are administered to those hosts at risk of N. meningitidis infection such as adults.

Pharmaceutical compositions are preferably in unit dosage form of about 0.001 to 100 μg/kg (antigen/body weight) and more preferably 0.01 to 10 μg/kg and most preferably 0.1 to 1 μg/kg 1 to 3 times with an interval of about −1 to 6 week intervals between immunizations.

Pharmaceutical compositions are preferably in unit dosage form of about 0.1 μg to 10 mg and more preferably 1 μg to 1 mg and most preferably 10 to 100 μg 1 to 3 times with an interval of about 1 to 6 week intervals between immunizations.

According to another aspect, there are provided pharmaceutical compositions comprising a liposome associated with polynucleotides encoding polypeptides characterized by the amino acid sequence comprising SEQ ID No: 2 or fragments or analogs thereof.

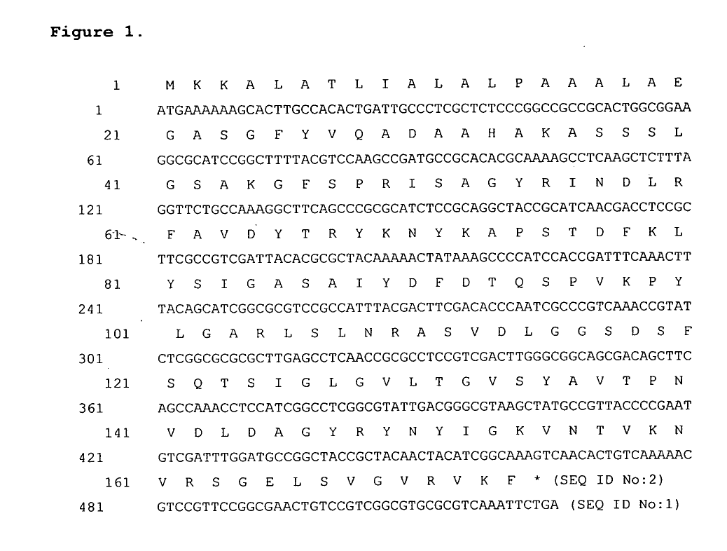

It will be appreciated that the polynucleotide sequences illustrated in FIG. 1 may be altered with degenerate codons yet still encode the polypeptides of the invention. Accordingly the present invention further provides pharmaceutical compositions comprising a liposome and polynucleotides which hybridize to the polynucleotide sequences herein above described (or the complement sequences thereof) having 90% identity between sequences. In a further embodiment, polynucleotides are hybridizable under stringent conditions i.e. having at least 95% identity. In a further embodiment, more than 97% identity.

Suitable stringent conditions for hybridation can be readily determined by one of skilled in the art (see for example Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.).

In a further embodiment, pharmaceutical compositions comprising a liposome associated with polynucleotides illustrated in SEQ ID NO: 1 or fragments or analogs thereof encoding polypeptides of the invention.

According to another aspect, there is provided a process for producing polypeptides of the invention by recombinant techniques by expressing a polynucleotide encoding said polypeptide in a host cell and recovering the expressed polypeptide product.

Alternatively, the polypeptides can be produced according to established synthetic chemical techniques i.e. solution phase or solid phase synthesis of oligopeptides which are ligated to produce the full polypeptide (block ligation).

General methods for obtention and evaluation of polynucleotides and polypeptides are described in the following references: Sambrook et al, Molecular Cloning: A Laboratory Manual, 2nd ed, Cold Spring Harbor, N.Y., 1989; Current Protocols in Molecular Biology, Edited by Ausubel F. M. et al., John Wiley and Sons, Inc. New York; PCR Cloning Protocols, from Molecular Cloning to Genetic Engineering, Edited by White B. A., Humana Press, Totowa, N.J., 1997, 490 pages; Protein Purification, Principles and Practices, Scopes R. K., Springer-Verlag, New York, 3rd Edition, 1993, 380 pages; Current Protocols in Immunology, Edited by Coligan J. E. et al., John Wiley & Sons Inc., New York.

The present invention provides a process for producing a polypeptide comprising culturing a host cell of the invention under conditions suitable for expression of said polypeptide.

For recombinant production, host cells are transfected with vectors which encode the polypeptides of the invention, and then cultured in a nutrient media modified as appropriate for activating promoters, selecting transformants or amplifying the genes. Suitable vectors are those that are viable and replicable in the chosen host and include chromosomal, non-chromosomal and synthetic DNA sequences e.g. bacterial plasmids, phage DNA, baculovirus, yeast plasmids, vectors derived from combinations of plasmids and phage DNA. The polypeptide sequence may be incorporated in the vector at the appropriate site using restriction enzymes such that it is operably linked to an expression control region comprising a promoter, ribosome binding site (consensus region or Shine-Dalgarno sequence), and optionally an operator (control element). One can select individual components of the expression control region that are appropriate for a given host and vector according to established molecular biology principles (Sambrook et al, Molecular Cloning: A Laboratory. Manual, 2nd ed, Cold Spring Harbor, N.Y., 1989; Current Protocols in Molecular Biology, Edited by Ausubel F. M. et al., John Wiley and Sons, Inc. New York). Suitable promoters include but are not limited to LTR or SV40 promoter, E. coli lac, tac or trp promoters and the phage lambda PL promoter. Vectors will preferably incorporate an origin of replication as well as selection markers i.e. ampicilin resistance gene. Suitable bacterial vectors include pET, pQE70, pQE60, pQE-9, pD10 phagescript, psiX174, pbluescript SK, pbsks, pNH8A, pNH16a, pNH18A, pNH46A, ptrc99a, pKK223-3, pKK233-3, pDR540, pRIT5 and eukaryotic vectors pBlueBacIII, pWLNEO, pSV2CAT, pOG44, pXT1, pSG, pSVK3, pBPV, pMSG and pSVL. Host cells may be bacterial i.e. E. coli, Bacillus subtilis, Streptomyces; fungal i.e. Aspergillus niger, Aspergillus nidulins; yeast i.e. Saccharomyces or eukaryotic i.e. CHO, COS.

Upon expression of the polypeptide in culture, cells are typically harvested by centrifugation then disrupted by physical or chemical means (if the expressed polypeptide is not secreted into the media) and the resulting crude extract retained to isolate the polypeptide of interest. Purification of the polypeptide from culture media or lysate may be achieved by established techniques depending on the properties of the polypeptide i.e. using ammonium sulfate or ethanol precipitation, acid extraction, anion or cation exchange chromatography, phosphocellulose chromatography, hydrophobic interaction chromatography, hydroxylapatite chromatography and lectin chromatography. Final purification may be achieved using HPLC.

The polypeptides may be expressed with or without a leader or secretion sequence. In the former case the leader may be removed using post-translational processing (see U.S. Pat. No. 4,431,739; U.S. Pat. No. 4,425,437; and U.S. Pat. No. 4,338,397) or be chemically removed subsequent to purifying the expressed polypeptide.

According to a further aspect, the pharmaceutical composition of the invention may be used in a diagnostic test for neisserial infection, in particular N. meningitidis infection.

Several diagnostic methods are possible, for example detecting N. meningitidis organism in a biological sample, the following procedure may be followed:
a) obtaining a biological sample from a host;
b) incubating an antibody or fragment thereof reactive with a pharmaceutical composition of the invention with the biological sample to form a mixture; and
c) detecting specifically bound antibody or bound fragment in the mixture which indicates the presence of N. meningitidis.

Alternatively, a method for the detection of antibody specific to a N. meningitidis antigen in a biological sample containing or suspected of containing said antibody may be performed as follows:
a) obtaining a biological sample from a host;
b) incubating a pharmaceutical composition of the invention with the biological sample to form a mixture; and
c) detecting specifically bound antigen or bound fragment in the mixture which indicates the presence of antibody specific to N. meningitidis.

One of skill in the art will recognize that this diagnostic test may take several forms, including an immunological test such as an enzyme-linked immunosorbent assay (ELISA), a radioimmunoassay or a latex agglutination assay, essentially to determine whether antibodies specific for the protein are present in an organism.

The DNA sequences encoding polypeptides of the invention may also be used to design DNA probes for use in detecting the presence of N. meningitidis in a biological sample suspected of containing such bacteria. The detection method of this invention comprises:
a) obtaining the biological sample from a host;
b) incubating one or more DNA probes having a DNA sequence encoding a polypeptide of the invention or fragments thereof with the biological sample to form a mixture; and
c) detecting specifically bound DNA probe in the mixture which indicates the presence of N. meningitidis bacteria.

The DNA probes of this invention may also be used for detecting circulating N. meningitidis i.e. N. meningitidis nucleic acids in a sample, for example using a polymerase chain reaction, as a method of diagnosing N. meningitidis infections. The probe may be synthesized using conventional techniques and may be immobilized on a solid phase, or may be labelled with a detectable label. A preferred DNA probe for this application is an oligomer having a sequence complementary to at least about 6 contiguous nucleotides of the N. meningitidis polypeptides of the invention. In a further embodiment, the preferred DNA probe will be an oligomer having a sequence complementary to at least about 15 contiguous nucleotides of the N. meningitidis polypeptides of the invention. In a further embodiment, the preferred DNA probe will be an oligomer having a sequence complementary to at least about 30 contiguous nucleotides of the N. meningitidis polypeptides of the invention. In a further embodiment, the preferred DNA probe will be an oligomer having a sequence complementary to at least about 50 contiguous nucleotides of the N. meningitidis polypeptides of the invention.

Another diagnostic method for the detection of N. meningitidis in a host comprises:
a) labelling an antibody reactive with a pharmaceutical composition of the invention with a detectable label;
b) administering the labelled antibody to the host; and
c) detecting specifically bound labelled antibody or labelled fragment in the host which indicates the presence of N. meningitidis.

A further aspect of the invention is the use of the pharmaceutical compositons of the invention as immunogens for the production of specific antibodies for the diagnosis and in particular the treatment of N. meningitidis infection. Suitable antibodies may be determined using appropriate screening methods, for example by measuring the ability of a particular antibody to passively protect against N. meningitidis infection in a test model. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal, or preferably monoclonal. It may be specific for a number of epitopes associated with the N. meningitidis polypeptides but is preferably specific for one.

According to one aspect, the present invention provides the use of an antibody for prophylaxis and/or treatment of N. meningitidis infections.

A further aspect of the invention is the use of the antibodies directed to the pharmaceutical compositions of the invention for passive immunization. One could use the antibodies described in the present application.

A further aspect of the invention is a method for immunization, whereby an antibody raised by a pharmaceutical composition of the invention is administered to a host in an amount sufficient to provide a passive immunization.

In a further embodiment, the invention provides the use of a pharmaceutical composition of the invention in the manufacture of a medicament for the prophylactic or therapeutic treatment of N. meningitidis infection.

In a further embodiment, the invention provides a kit comprising a pharmaceutical composition of the invention for detection or diagnosis of N. meningitidis infection.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE 1

This example illustrates the 3-D model representing the NspA protein.

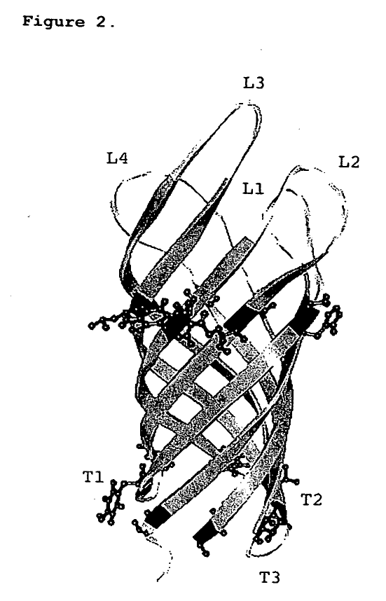

A 3-D model of the meningococcal NspA protein was developed based on the crystal structure of the refolded E. coli OmpA (PDB: 1QJP) [Pautsch, A. and GE Schulz, J. Mol. Biol. 298, p. 273 (2000)] using Swiss-Pdb Viewer [Guex, N. and MC Peitsch, Electrophoresis, 18, p. 2714 (1997)] and the NspA amino acid sequence presented in FIG. 1. This sequence as well as other NspA sequences were originally presented in PCT/WO/96/29412. The 3-D NspA model is presented in FIG. 2. The alignment between the prediction target (NspA sequence) and the template (1QJP, OMPA sequence) was achieved using secondary structure prediction (PSIPRED), profile library search (FUGUE), position specific iterated BLAST (PSI-BLAST) and beta-strands amphipaticity determination [Shi J. et al. J. Mol. Biol., 310, p. 243 (2001); McGuffin L. T. et al. Bioinformatics, 16, p. 404. (2000); Altschul S. F. et al. Nucleic Acids Res., 25, p. 3389 (1997)]. From this model, it was possible to localize each region of the protein and to classify them as periplasmic turn (T), membrane embedded region (M) and surface-exposed loop (L). As reported previously, the first 18 amino acid residues represent the secretion signal, which is cleaved in the mature polypeptide [Martin et al. J. Exp. Med., 185, p. 1173 (1997)]. Three sharp turns, which extend outside the membrane facing the periplasmic region of the bacteria, were localized between residues 55-58 (T1), 92-96 (T2) and 137-140 (T3). The internal core of the NspA protein, which is embedded in the meningococcal membrane, is made of 8 antiparallel transmembrane β-strands forming a β-barrel. These transmembrane β-strands were determined to be located between the amino acid residues 24-33 (M1), 45-54 (M2), 59-67 (M3), 81-91 (M4), 97-107 (M5), 126-136 (M6), 141-150 (M7), and 164-173 (M8) respectively. Finally, four regions, which were determined to be exposed at the surface of the meningococcal cells, were localized between the amino acid residues 34-44 (L1), 68-80 (L2), 108-125 (L3), and 151-163 (L4) respectively. The immunological confirmation of this model is presented in Example 5.

### EXAMPLE 2

This example illustrates the generation of ANspA N. meningitidis mutant strain.

To generate a meningococcal mutant strain not expressing the NspA protein, the gene was inactivated using the transposon mini-Tn10 (Kan^{r}), which is inserted in the phage vector λ1105 [Way et al. Gene, 32, p. 369 (1984); Kleckner et al. Methods Enzymol., 204, p. 139 (1991)]. The plasmid pN2202, which contained the nspA gene [Martin et al. J. Exp. Med., 185, p. 1173 (1997)], was used to transform the E. coli strain W3110 [F−, hsdR−, hsdM+, thy−, IN(rrnD-rrnE)1λ−, mcrA+, mcrB+, (rₖ+, mₖ+), mrr+, su^{o}]. The recombinant E. coli strain was then infected with the phage vector λ1105, and the culture was plated on LB agar plates containing 25 μg/ml ampicillin and 25 μg/ml of kanamycin and incubated overnight at 37° C. Only the bacteria, which contained the mini-Tn10 transposon on either the chromosome or the pN2202 plasmid will grow on the selective media. The recombinant pN2202 plasmid was purified using QIAgen plasmid purification kit from selected colonies. These purified plasmids were then used to transform E. coli strain JM109 (e14⁻ (mcrA) recA1 endA1 gyrA96 thi-1 hsdR17 (rₖ₋ mₖ₊) supE44 relA1 Δ(lac-proAB) (F′ traD36 proAB lacI^{q}ZΔM15)) and the bacterial suspension was again plated on selective media. Only the bacteria containing the recombinant pN2202 plasmid, identified as pN2202ΔnspA, with the mini-Tn10 transposon were able to grow after this second round of selection. Immunoblots confirmed that these recombinant E. coli did not produce the NspA protein. The plasmid was purified from one of the E. coli recombinant strain, and the presence of the mini-Tn10 transposon in the nspA gene was confirmed by sequencing. It was determined that the 1.8 kb mini-Tn10 was inserted immediately after nucleotide 221 in the nspA gene contained on the plasmid pN2202AnspA. The plasmid pN2202AnspA was then used to transform the meningococcal strain 608B according to the following protocol. The optical density (λ=620 nm) of the bacterial suspension of meningococcal strain 608B grown in heart infusion broth with 10 mM MgCl₂ was adjusted to ˜0.25. A volume of 10 μl of purified plasmid pN2202AnspA was added to 1 ml of the adjusted meningococcal cell suspension and incubated for 3 h at 37° C. in the presence of 5% CO₂. After this incubation period, the meningococcal cells were plated on chocolate agar plates containing 25 μg/ml of kanamycin. The lack of expression of the NspA protein was confirmed by immunoblotting and flow cytofluorometry assays. As expected, the NspA-specific MAb Me-7 as well as rabbit and mouse hyperimmune sera did not react with the 608BAnspA mutant strain, while they recognized the wild type meningococcal 608B strain.

### EXAMPLE 3

This example illustrates the generation of NspA-specific monoclonal antibodies.

To generate MAbs directed against native NspA, female Balb/c ice were immunized with an outer membrane preparation extracted from the serogroup B N. meningitidis strain 608B [B:2a:P1.2:L3] [Martin et al. J. Exp. Med., 185, p. 1173 (1997)]. The lithium chloride extraction used to obtain this outer membrane preparation was performed in a manner previously described by the inventors [Brodeur et al. Infect. Immun., 50, p. 265 (1985)]. Mice were injected intramuscularly (IM) three times with 20 μg of outer membrane preparation at three-week intervals in the presence of QuilA adjuvant (Cedarlane Laboratories, Hornby, Ont., Canada). The fusion protocol used to generate the hybridoma cell lines was described previously by the inventors [Hamel et al. J. Med. Microbiol., 25, p. 2434 (1987)]. The class and subclass of the MAbs were determined by ELISA as previously reported [Martin et al. J. Exp. Med., 185, p. 1173 (1997)].

The specificity of the MAbs was determined by ELISA using purified recombinant NspA protein, outer membrane preparations extracted from N. meningitidis wild type strain 608B and the 608BΔnspA mutant strain and the data are presented in Table 1. The ELISA were performed as described previously. [Martin et al. J. Exp. Med., 185, p. 1173 (1997)]. MAb Me-7, which was described previously in PCT/WO/96/29412 was used as a positive control and MAb P2-4, which is specific from Haemophilus influenzae P2 outer membrane protein was used as negative control [Cadieux et al. Infect. Immun., 67, p. 4955, (1999)]. All MAbs reacted strongly with the purified recombinant NspA and with outer membrane preparation extracted from the meningococcal wild type 608B strain, but they did not recognize the meningococcal 608BΔnspA mutant strain.

TABLE 1
Reactivity of NspA-specific MAbs

| | | Reactivity¹ of MAbs with | | |
|---|---|---|---|---|
| | | | Wild type | |
| | | Recombinant | outer | ΔNspA outer |
| Mab ID | Isotype | NspA | membrane | membrane |
| Me-7 | IgG2a | + | + | − |
| Me-9 | IgG3 | + | + | − |
| Me-10 | IgG2a | + | + | − |
| Me-11 | IgG2b | + | + | − |
| Me-12 | IgG2b | + | + | − |
| Me-13 | IgG2a | + | + | − |
| Me-14 | IgG2a | + | + | − |
| Me-15 | IgG2a | + | + | − |
| Me-16 | IgG1 | + | − | − |
| Me-17 | IgG2a | + | + | − |
| Me-18 | IgG2a | + | + | − |
| Me-19 | IgG2a | + | + | − |
| Me-20 | IgG3 | + | + | − |
| Me-21 | IgG2a | + | + | − |
| Me-22 | IgG2a | + | + | − |
| P2-4 | IgG2a | − | − | − |

¹The reactivity of the Mabs was evaluated by ELISA using 0.5 μg/ml of purified recombinant NspA protein, 2.5 μg/ml of OMP from wild type 608B meningococcal strain or from 608BΔnspA strain as coating antigen.

Exposure of NspA at the surface of intact meningococcal cells was studied using a cytofluorometric assay. Meningococci were grown in brain heart infusion (BHI) broth containing 0.25% dextrose at 37° C. in a 8% CO₂ atmosphere up to an optical density λ=490 nm) of 0.500 (˜10⁸ CFU/ml). NspA-specific MAbs or control Mab were then added and allowed to bind to the cells, which were incubated for 2 h at 4° C. with rotation. Samples were washed twice in blocking buffer [phosphate-buffered saline (PBS) containing 2% bovine serum albumin (BSA)], and then 1 ml of goat fluorescein (FITC)-conjugated anti-mouse specific IgG (H+L) diluted in blocking buffer was added. After an additional incubation of 60 min at room temperature with rotation, samples were washed twice in PBS buffer and fixed with 0.3% formaldehyde in PBS buffer for 18 h at 4° C. Cells were kept in the dark at 4° C. until analyzed by flow cytometry (Epics® XL; Beckman Coulter, Inc.).

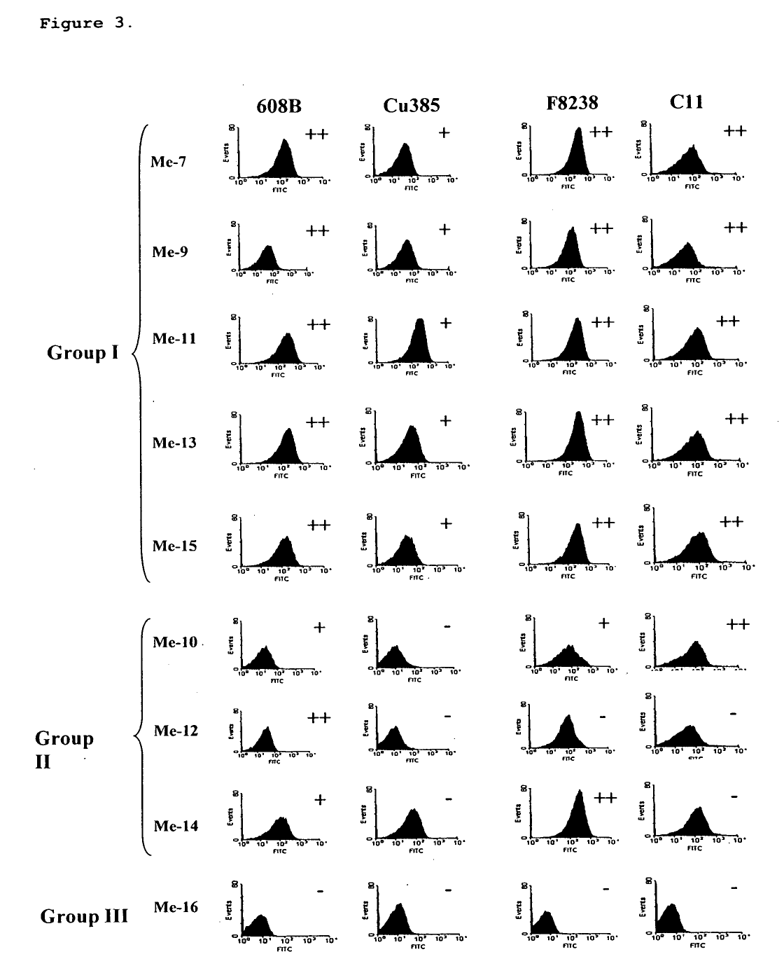

FIG. 3 presents the attachment of 9 representative NspA-specific MAbs at the surface of two serogroup B (608B) [Martin et al. J. Exp. Med., 185, p. 1173 (1997)] and CU385 [Moe et al. Infect. Immun., 67, p. 5664, (1999)], one serogroup A (F8238) [Maslanka et al., Clin. Diagn. Lab. Immunol., 4, p. 156 (1997)] and one serogroup C (C11) [Maslanka et al., Clin. Diagn. Lab. Immunol., 4, p. 156 (1997)] meningococcal strains. For each MAb, the concentration was adjusted at 1 μg/mL and early log phase meningococcal cells were used to perform the cytofluorometry assay. None of these MAbs reacted with the 608BΔnspA mutant strain from which the nspA gene was inactivated by the insertion of a transposon (See Example 2 for a description of the mutant strain). This result indicated that none of these MAbs attached non-specifically at the surface of live meningococcal cells.

According to the level of attachment to intact meningococcal cells, the NspA-specific MAbs were classified in three groups (FIG. 3). In the first group, MAbs such as Me-7, Me-9, Me-11, Me-13 and Me-15 attached efficiently at the cell surface of the four strains tested, indicating that their epitopes are located on surface-exposed regions of the protein. The binding of MAbs, such as Me-10, Me-12 and Me-14, which were classified in the second group, was more variable since they recognized their corresponding epitopes at the surface of one or two strains out of the four tested. Finally, MAbs such as Me-16, which did not bind to any intact meningococcal cells were classified in the third group. Immunoblots clearly indicated that the MAbs in the latter group reacted well with purified NspA when it was not inserted into the meningococcal outer membrane (data not shown).

Globally these binding data suggested that some epitopes present on the NspA are exposed and accessible to specific antibodies at the cell surface of serologically distinct meningococcal cells, while other epitopes are accessible to antibodies on a limited number of strains. Since the NspA protein is highly conserved and is produced by all strains tested to date, the lack of binding of group II MAbs to certain meningococcal strains is most probably not related to amino acid variation, or lack of protein expression. One might postulate that other antigens present at the meningococcal cell surface might mask the epitopes recognized by the MAbs in the second group, or that the tertiary structure of the protein might be slightly different in these strains thus preventing the binding of antibodies to certain epitopes. It was reported that the polysaccharide capsule could shield the NspA epitopes and prevent binding of antibodies to meningococcal strains that produce large amount of polysaccharides [Moe et al. Infect. Immun., 67, p. 5664, (1999)]. However, the relationship between polysaccharide production, lack of binding and bactericidal activity of NspA-specific antibodies was not clearly established. Indeed, anti-NspA antibodies could bind to the surface and kill a meningococcal strain, which was determined to be a high polysaccharide producer, while a low-producer strain was negative for surface binding and resistant to bactericidal activity. Considering this latter observation, one might postulate that other mechanisms, such as conformational changes, may also explain the lack of binding and bactericidal activity observed for certain MAbs.

MAbs classified in group I, which recognized their specific epitopes at the surface of all four strains, were found to be bactericidal against the four meningococcal strains tested (FIG. 3). For group I MAbs, the data suggest a correlation between surface binding and the bactericidal activity. However, it is difficult to establish any relation for the MAbs classified in group II. As an example, the meningococcal strain C11 was resistant to the bactericidal-activity of MAbs Me-12 and Me-14 even though it was positive for surface binding.

### EXAMPLE 4

This example describes the cloning of modified nspA gene products by polymerase chain reaction (PCR), and the expression of these gene products in E. coli.

In order to characterize the NspA surface-exposed epitopes, seven modified NspA proteins have been designed (Table 2). Gene fragments to be included in the modified nspA genes designated Nm14, Nm16, Nm17, and Nm20 were amplified by PCR (DNA Thermal Cycler GeneAmp PCR system 2400 Perkin Elmer) from nspA or Nm19 (for Nm20) gene cloned into pURV vector described in patent PCT/WO/96/29412 using pairs of oligonucleotide primers that contained base extensions for the addition of restriction sites (Table 3 and 4) according to standard methods. PCR products were purified from agarose gel using a QIAquick gel extraction kit from QIAgen following the manufacturer's instructions, and digested with restriction endonucleases. The pURV vector was digested with the endonucleases NdeI and NotI and purified from agarose gel using a QIAquick gel extraction kit from QIAgen. The digested PCR products corresponding to a given modified nspA gene were ligated into pURV-NdeI-NotI vector for the generation of a modified nspA gene. The ligated product was transformed into E. coli strain DH5α [F⁻φ80dlacZΔM15 Δ(lacZYA-argF)U169 endA1 recA1 hsdR17(rₖ⁻mₖ⁺) deoR thi-1 phoA supE44 λ⁻gyrA96 relA1] (Gibco BRL, Gaithersburg, Md.) according to the manufacturer's recommendations. Recombinant plasmids containing the modified nspA gene fragments were purified using a QIAgen plasmid kit and their DNA insert was sequenced (Taq Dye Deoxy Terminator Cycle Sequencing kit, ABI, Foster City, Calif.).

To complete the modified proteins Nm14, Nm16, and Nm17, Nm20 and to generate the protein Nm3, mutagenesis experiments using the Quickchange Site-Directed Mutagenesis kit from Stratagene and the oligonucleotides described in Table 5 were performed according to the manufacturer's recommendations. The Table 6 presents the modifications on modified nspA genes generated by site-directed mutagenesis.

In order to generate the protein Nm18, the N-terminal fragment was amplified by PCR using the oligonucleotide primers DMAR839 and DMAR1159 that contained base extensions for the addition of restriction sites (Table 4) and digested as described above. The C-terminal fragment was generated using the oligonucleotide primers DMAR1157 and DMAR1158 as adaptor after annealing of these primers according to standard methods. The ligation into pURV-NdeI-NotI vector and the tranformation into E. coli strain DH5α were performed as described above. Recombinant plasmid containing the modified nspA gene fragment was purified using a QIAgen plasmid kit and its DNA insert was sequenced (Taq Dye Deoxy Terminator Cycle Sequencing kit, ABI, Foster City, Calif.).

To generate the Nm19 molecule, the modified genes Nm16 and Nm18 were digested with the endonucleases NdeI-SalI and SalI-NotI, respectively. The fragments were purified from agarose gel using a QIAquick gel extraction kit from QIAgen, and ligated into pURV-NdeI-NotI vector. The recombinant plasmid containing the modified gene Nm19 was purified using a QIAgen plasmid kit and its. DNA insert was sequenced (Taq Dye Deoxy Terminator Cycle Sequencing kit, ABI, Foster City, Calif.).

Each of the resultant plasmid constructs was used to transform by electroporation (Gene Pulser II apparatus, BIO-RAD Labs, Mississauga, Ontario, Canada) E. coli strain BL21 (F⁻ ompT hSdS_{B}(r⁻_{B}m⁻_{B}) gal dcm) (Novagen). This recombinant strain was inoculated in LB broth (Gibco BRL) containing 40 μg/ml of kanamycin, and was first incubated at 37° C. for approximately 1.5 h with agitation (OD_{600 nm}=0.6) after which time the temperature was increased to 39° C. for an additional 1.5 h in order to induce the production of the recombinant protein. In order to characterize the surface-exposed epitopes, the NspA-specific Mabs were tested using cytofluorometry assay, as described at Example 5, against the E. coli cells obtained after the induction period.

TABLE 2
List of the modified nspA genes.

| Gene/Protein | |
|---|---|
| designation | Characteristics |
| Nm3 | G exchange for A (position 115) |
| | D exchange for N (position 118) |
| Nm14 | Loop 2 deleted (AA 67 to AA 79) |
| Nm16 | Loop 1 deleted (AA 36 to AA 43) |
| Nm17 | Loop 3 deleted (AA 111 to AA 122) |
| Nm18 | Loop 4 deleted (AA 152 to AA 163) |
| Nm19 | Loop 1 (AA 36 to AA 43) and 4 (AA 152 to AA 163) |
| | deleted |
| Nm20 | Loop 1 (AA 36 to AA 43), loop 2 (AA 67 to AA |
| | 79), and 4 (AA 152 to AA 163) deleted |

TABLE 3
List of PCR oligonucleotide primer pairs designed for
the generation of modified nspA genes listed in Table 2.

| | | Corresponding position of |
|---|---|---|
| Gene/Protein | PCR-primer | the gene fragment on the |
| designation | identification | modified protein molecule |
| Nm3 | DMAR837-DMAR838 | Complete |
| Nm14 | DMAR839-DMAR937 | N-terminal |
| | DMAR840-DMAR938 | C-terminal |
| Nm16 | DMAR839-DMAR1149 | N-terminal |
| | DMAR840-DMAR1152 | C-terminal |
| Nm17 | DMAR839-DMAR1153 | N-terminal |
| | DMAR840-DMAR1154 | C-terminal |
| Nm18 | DMAR839-DMAR1159 | N-terminal |
| | DMAR1157-DMAR1158 | C-terminal |
| Nm20 | DMAR839-DMAR1160 | N-terminal |
| | DMAR840-DMAR1161 | C-terminal |

TABLE 4

List of PCR oligonucleotide primers designed for
the generation of modified nspA genes listed in
Table 2.

| | | Restriction |
|---|---|---|
| Primer | Sequence 5′-3′ | site |
| DMAR839 | ggaattccatatgaaaaaagcacttgccac | NdeI |
| DMAR840 | ataagaatgcggccgctcagaatttgacgcg | NotI |
| | cac | |
| DMAR937 | tcgaggtacccgtgtaatcgacggcgaagc | KpnI |
| DMAR938 | tcgaggtaccctttacagcatcggcgcg | KpnI |
| DMAR1149 | tcgaggtacctgtttttgcgtgtgcggcatc | KpnI |
| | gg | |
| DMAR1152 | tcgaggtaccaaaggcttcagcccgcgc | KpnI |
| DMAR1153 | atatgggcccggcgcggttgaggctcaagc | ApaI |
| DMAR1154 | atatgggccctccaacacctccatcggcctc | ApaI |
| | ggcg | |
| DMAR1157 | cgataatggcgaactgtccgtcggcgtgcgc | — |
| | gtcaaattctgagc | |
| DMAR1158 | ggccgctcagaatttgacgcgcacgccgacg | — |
| | gacagttcgccattatcgggcc | |
| DMAR1159 | atatgggcccgtagttgtagcggtagccggc | ApaI |
| DMAR1160 | tcgaggtacccgtgtaatcgacggcgaagcg | KpnI |
| DMAR1161 | tcgaggtaccctttacagcatcggcgcgtcc | KpnI |

TABLE 5

List of PCR oligonucleotide primer sets used for
site-directed mutagenesis on modified nspA genes

| Gene/Protein | Primer | Primer SEQUENCE |
|---|---|---|
| designation | Identification | 5′--->3′ |
| Nm3 | DMAR837 | ccgcgcctccgtcgacttggc |
| | | cggcagcaacagcttcagcca |
| | | aac |
| | DMAR838 | gtttggctgaagctgtcgctg |
| | | ccgcccaagtcgacggaggcg |
| | | cgg |
| Nm14 | DMAR941 | cgcttcgccgtcgattacacg |
| | | ggtaacctttacagcatcggc |
| | | gcg |
| | DMAR942 | cgcgccgatgctgtaaaggtt |
| | | acccgtgtaatcgacggcgaa |
| | | gcg |
| Nm16 | DMAR1150 | gcgcgggctqaagcctttgtt |
| | | acctgtttttgcgtgtgcggc |
| | DMAR1151 | gccgcacacgcaaaaacaggt |
| | | aacaaaggcttcagcccgcgc |
| Nm17 | DMAR1155 | ttgaqcctcaaccgcgccggg |
| | | ggctccaacacctccatcggc |
| | | ctc |
| | DMAR1156 | gaggccgatggaggtgttgga |
| | | gcccccggcgcggttgaggct |
| | | caa |
| Nm20 | DMAR1162 | ggacgcgccgatgctgtaaag |
| | | gttacccgtgtaatcgacggc |
| | | gaa |
| | DMAR1163 | ttcgccgtcgattacacgggt |
| | | aacctttacagcatcggcgcg |
| | | tcc |

TABLE 6

List of modifications on modified nspA gene
products generated by site-directed mutagenesis

| | Molecule | |
|---|---|---|
| Gene/Protein | used for | |
| designation | mutagenesis | DNA modifications¹ |
| Nm3 | nspA | 341-TGGCCGGCAGCAACA-355 |
| Nm14 | Nm14 | 201-GGGTAACCTT-210 |
| Nm16 | Nm16 | 111-TAACAAAGGC-120 |
| Nm17 | Nm17 | 331-GGGGGCTCCA-340 |
| Nm20 | Nm20 | 181-ACGGGTAACC-190 |

¹The underlined amino acid residues represent the modification in DNA sequence.

### EXAMPLE 5

This example illustrates the localization of the epitopes recognized by the MAbs on the NspA protein.

To localize the epitopes recognized by the NspA-specific Mabs and to confirm the NspA model presented in Example 1, the surface binding of these MAbs was evaluated by flow cytometry using recombinant E. coli strains that were producing the modified NspA proteins described in Example 4 and by ELISA with overlapping synthetic peptides covering the NspA protein.

The epitopes recognized by group III MAbs, such as Me-16, were easily located using overlapping 15- to 20-amino-acid-residue synthetic peptides covering the full-length of the NspA protein. These peptides were presented in the patent PCT/WO/96/29412. As an example, MAb Me-16 was found by ELISA to react with two separate peptides located between residues 41-55 (GSAKGFSPRISAGYR) and 141-150 (VDLDAGYRYNYIGKV). Closer analysis revealed that these two peptides shared the AGYR residues, which are underlined in the peptide sequences. According to the NspA model (FIG. 2), these two regions are embedded inside the meningococcal outer membrane and as expected, antibodies directed against these regions did not attach to intact meningococcal cells (FIG. 3).

MAbs that were classified in groups I and II did not react with any of these peptides. These results suggest that these MAbs are directed against conformationally restricted epitopes. These epitopes can be easily modified or lost during the production, purification and formulation of meningococcal outer membrane protein as observed with the PorA [Jansen et al. FEMS Immunol. Med. Microbiol., 27, p. 227 (2000); Peeters et al. Vaccine, 17, p. 2702 (1999): Niebla et al. Vaccine, 19; p. 3568 (2001)] and Opc proteins [Carminate et al. Biotechnol. Appl. Biochem., 34, p. 63, (2001)]. Antibodies raised against these incorrectly folded proteins are of limited use since they often are biologically less active. To localize these conformational epitopes, a series of modified NspA proteins, where different combinations of surface-exposed loops were deleted or mutated, were constructed (Example 4). To maintain the conformation of these modified NspA proteins, they were produced in E. coli membranes. The reactivity of selected MAbs with these modified NspA proteins was evaluated by cytofluorometric assays. The attachement of the MAbs to the cells are presented in Table 7 as binding indexes that were calculated as the median fluorescence values obtained after labelling the cells with NspA-specific MAbs divided by the fluorescence value obtained for a control MAb. A fluorescence value of 1 indicated that there was no binding of antibodies at the surface of intact cells. The presence of these modified NspA proteins in the outer membrane of recombinant E. coli cells was confirmed by immunoblots using MAb Me-16. As presented above, MAb Me-16 recognized a linear epitope, which is not sensitive to con-formational changes. This epitope is located in the transmembrane portion of the protein, not on the surface exposed loops. Immunoblots revealed that MAb Me-16 reacted with all the modified NspA proteins confirming that the recombinant E. coli cells were producing these proteins in their outer membranes.

MAbs classified in group II recognized epitopes on the NspA protein that were highly sensitive to conformational changes induced by either deletions or mutations to the four surface-exposed loops. Binding of MAb Me-10 to recombinant E. coli cells producing the modified NspA in their membranes was highly sensitive to any modification at any of the 4 surface-exposed loops. This result suggests that the epitope recognized by this MAb is surface-exposed, conformational and that the binding of this MAb can be prevented by minor structural modifications to the NspA protein. Contrary to the binding specificity observed for MAb Me-10, deletion of loop 4 (Nm18) did not prevent the binding of MAbs Me-12 and Me-14 to the recombinant E. coli cells.

With the exception of MAb Me-7, MAbs classified in group I are directed against conformational epitopes that needed both loops 2 and 3 to be correctly presented at the cell surface. Mutation to (Nm3), or deletion (Nm14, Nm17) of one of these two loops significantly reduced, or completed prevented the binding of MAbs Me-11, Me-17 and Me-19 to recombinant E. coli cells. On the contrary, deletion of loop 1 (Nm16), loop 4 (Nm18) and loops 1 and 4 (Nm19) did not significantly reduce the binding of these MAbs to recombinant E. coli cells. These results suggest that the epitopes recognized by these MAbs need both loops 2 and 3 to be correctly presented at the surface of intact cells.

The reactivity of MAb Me-7 with these modified NspA proteins clearly indicated that its corresponding epitope is located only on loop 3. Indeed, binding of MAb Me-7 to recombinant E. coli cells producing either a mutated NspA protein (Nm3), or a protein without deleted loop 3 (Nm17) was prevented. For Nm3 NspA protein, the glycine (G) and aspartic acid (D) at position 115 and 118 were respectively replaced by an alanine (A) and an asparagine (N). The lack of reactivity of MAb Me-7 with recombinant E. coli cells that produced Nm3 indicated that the specific epitope is located at the tip of loop 3.

The results presented in this example demonstrate that at least loops 2, 3 and 4 are exposed at the surface of the bacteria and thus confirm that the 3-D NspA model presented in Example 1 is adequate. Surface-exposure of loop 1 was not confirmed since no MAb specific for that portion of the protein was available. More importantly, these data clearly indicate that most bactericidal NspA-specific MAbs are directed against conformational epitopes located on loop 2 and/or loop 3. One can speculate that vaccination with incorrectly folded NspA protein could prevent the induction of antibodies directed against these conformational epitopes and thus could reduce the protective potential of this protein.

TABLE 7
Evaluation of the binding of NspA-specific MAbs to
recombinant E. coli cells expressing different modified NspA
proteins in their outer membrane.

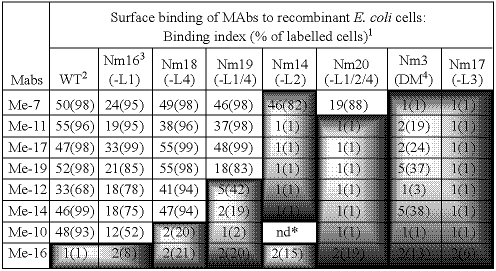

¹The binding index was calculated as the median fluorescence value obtained after labelling the cells with NspA-specific MAb divided by the fluorescence value obtained for a control MAb. A fluorescence value of 1 indicated that there was no binding of antibodies at the surface of intact cells. Boxes with a low index are shaded.
²Recombinant E. coli cells expressing the wild type NspA protein in their outer membrane.
³Name of the modified NspA protein (deletion)
⁴DM; double mutation on loop 3
*nd: not determined

### EXAMPLE 6

This example illustrates the method used for extracting lipids from bacterial cells.

Complex lipid mixtures were extracted from E. coli, N. meningitidis, and N. lactamica in order to generate liposome formulations from bacterial origin.

The following method was used to generate the complex lipid mixtures used to generate the liposome formulations presented in Example 7.

Bacteria were grown overnight in BHI broth at 37° C. in presence of 8% CO₂ (175 rpm). Cells were collected by centrifugation and the pellet was suspended in 6.7 ml of methanol per gram of cells (wet weight). This bacterial suspension was sonicated in an ice bath twice using a Sonic dismembrator 500 (Fisher Scientific) with a microtip probe adjusted at 8. This suspension was then heated at 65° C. for 30 min. After this incubation period, 2 volumes of chloroform were added to the suspension and agitated for 1 h at room temperature. The suspension was filtred through Whatman No. 4 filter. The filtrate was transferred in a teflon tube and 0.2 volume of saline solution (NaCl 0.6% (w/v)) was then added. After centrifugation, the upper phase and the precipitate at the interface were discarded. The lower phase was extracted with one volume of chloroform:methanol:saline solution (3:48:47) at least four times or until there was no more precipitate at the interface. After the final extraction, the lower organic phase was dried in a rotatory evaporator (Rotavapor, Buchi, Switzerland). The dried phospholipids were stored at −80° C. or resuspended in a solution of chloroform:methanol (2:1).

### EXAMPLE 7

This example illustrates the incorporation of recombinant NspA into different liposome formulations.

Liposomes were prepared using a dialysis method. Liposomes were prepared with different synthetic (see list 1 in this Example) or bacterial phospholipids with or without cholesterol, which were combined at different ratios. Some liposome formulations were also prepared with the adjuvant monophosphoryl lipid A (MPLA, Avanti polar lipids, Alabaster, Ala.) at 600 μg/ml. NspA protein was first precipitated in 99% ethanol (vol/vol) and denatured in 1 ml of PBS buffer containing 1% (wt/vol) of SDS (Sigma chemical), and heated at 100° C. for 10 min. The solution was diluted with 1 ml of PBS buffer containing 15% (wt/vol) of n-octyl β-D-glucopyranoside (OG, Sigma) and incubated at room temperature for 3 h. Lipids were dissolved in a chloroform:methanol solution (2:1) in a round bottom glass flask and dried using a rotatory evaporator (Rotavapor, Buchi, Switzerland) to achieve an even film on the vessel. The above protein-detergent solution was then added to the lipid film and mixed gently until the film was dissolved. The solution, after mixing, was slightly opalescent in appearance. The solution was then extensively dialysed against PBS buffer (pH 7.4) to remove detergent and to induce liposome formation. After dialysis, the resulting milky solution was sequentially extruded through 1000, 400, 200, and 100 nm polycarbonate filters using a stainless steel extrusion device (Lipex Biomembranes, Vancouver, Canada). The recombinant NspA not incorporated into the liposome was removed by centrifugation at 20000 g for 15 min at 4° C. The liposome solution was centrifuged at 250000 g for 1 h at 4° C. and the pellet was suspended with PBS buffer containing 0.3 M of sucrose. Vesicle size and homogeneity were evaluated by quasi-elastic light scattering with a submicron particles analyzer (model N4 Plus, Beckman Coulter). Using this apparatus, it was estimated that the liposome size in the different preparations was approximately 100 nm. All liposome preparations were sterilized by filtration through a 0.22 μm membrane and stored at −80° C. until used. The amount of recombinant protein incorporated in the liposome was estimated by MicroBCA (Pierce, Rockford, Ill.) after protein extraction of NspA-liposome preparations with chloroform:methanol solution (2:1) as described by Wessel and Flugge (Anal. Biochem. 1984, 138:141-143.)

Gel filtration and rapid dilution were used as alternate methods to induce the formation of NspA liposome. For the gel filtration method, the NspA-OG-SDS-lipids solution was applied directly on top of a Sephadex G-50 (column size: 2×20 cm, Pharmacia) or a P-6 (column size: 2×20 cm, Bio Rad) size exclusion chromatography/desalting column and eluted with PBS buffer at a flow rate of 2.5 ml/min. Fractions containing both protein and lipids were pooled, extruded, centrifuged, and the vesicle sizes were evaluated as described above. All preparations were sterilized through a 0.22 μm membrane and stored at −80° C. until used.

For rapid dilution method, a lipid film was prepared in a round bottom glass flask as described above. This lipid film was dissolved with a phosphate buffered solution (10 mM, 70 mM NaCl, pH 7.2) containing 1% triton X-100 and 750 μg/ml of NspA protein. Lipid-detergent-protein solution was then diluted drop-wise (1 drop/sec), with constant stirring, by the addition of 11 volumes of phosphate buffer. After dilution, the solution was kept at room temperature for 30 min with agitation. The recombinant NspA not incorporated into the liposome was removed by centrifugation and the liposome solution was ultracentrifuged as described above. Finally, the liposome pellet was suspended with PBS buffer containing 0.3 M sucrose. Vesicle size and homogeneity were evaluated as described above. All preparations were sterilized through a 0.22 μm membrane and stored at −80° C. until used.

List 1. Partial list of synthetic lipids used to prepare NspA-liposome preparations.

1,2-Dilauroyl-sn-Glycero-3-Phosphate (DLPA), Dimyristoyl-sn-Glycero-3-Phosphate (DMPA), 1,2-Dipalmitoyl-sn-Glycero-3-Phosphate (DPPA), 1,2-Distearoyl-sn-Glycero-3-Phosphate (DSPA), 1,2-Dioleoyl-sn-Glycero-3-Phosphate (DOPA), 1-Palmitoyl-2-Oleoyl-sn-Glycero-3-Phosphate (POPA), 1,2-Dilauroyl-sn-Glycero-3-Phosphocholine (DLPC), 1,2-Ditridecanoyl-sn-Glycero-3-Phosphocholine, 1,2-Dimyristoyl-sn-Glycero-3-Phosphocholine (DMPC), 1,2-Dipentadecanoyl-sn-Glycero-3-Phosphocholine, 1,2-Dipalmitoyl-sn-Glycero-3-Phosphocholine (DPPC), 1,2-Diheptadecanoyl-sn-Glycero-3-Phosphocholine, 1,2-Distearoyl-sn-Glycero-3-Phosphocholine (DSPC), 1,2-Dimyristoleoyl-sn-Glycero-3-Phosphocholine, 1,2-Dipalmitoleoyl-sn-Glycero-3-Phosphocholine, 1,2-Dioleoyl-sn-Glycero-3-Phosphocholine (DOPC), 1-Myristoyl-2-Palmitoyl-sn-Glycero-3-Phosphocholine, 1-Myristoyl-2-Stearoyl-sn-Glycero-3-Phosphocholine, 1-Palmitoyl-2-Myristoyl-sn-Glycero-3-Phosphocholine, 1-Palmitoyl-2-Stearoyl-sn-Glycero-3-Phosphocholine, 1-Palmitoyl-2-Oleoyl-sn-Glycero-3-Phosphocholine (POPC), 1-Palmitoyl-2-Linoleoyl-sn-Glycero-3-Phosphocholine, 1,2-Dilauroyl-sn-Glycero-3-Phosphoethanolamine (DLPE), 1,2-Dimyristoyl-sn-Glycero-3-Phosphoethanolamine (DMPE), 1,2-Dipalmitoyl-sn-Glycero-3-Phosphoethanolamine (DPPE), 1,2-Dipalmitoleoyl-sn-Glycero-3-Phosphoethanolamine, 1,2-Distearoyl-sn-Glycero-3-Phosphoethanolamine (DSPE), 1,2-Dioleoyl-sn-Glycero-3-Phosphoethanolamine (DOPE), 1-Palmitoyl-2-Oleoyl-sn-Glycero-3-Phosphoethanolamine (POPE), 1,2-Dilauroyl-sn-Glycero-3-[Phospho-RAC-(1-glycerol)] (DLPG), 1,2-Dimyristoyl-sn-Glycero-3-[Phospho-RAC-(1-glycerol)] (DMPG), 1,2-Dipalmitoyl-sn-Glycero-3-[Phospho-RAC-(1-glycerol)] (DPPG), 1,2-Distearoyl-sn-Glycero-3-[Phospho-RAC-(1-glycerol)] (DSPG), 1,2-Dioleoyl-sn-Glycero-3-[Phospho-RAC-(1-glycerol)] (DOPG), 1-Palmitoyl-2-Oleoyl-sn-Glycero-3-[Phospho-RAC-(1-glycerol)] (POPG), 1,2-Dilauroyl-sn-Glycero-3-[Phospho-L-Serine] (DLPS), 1,2-Dimyristoyl-sn-Glycero-3-[Phospho-L-Serine] (DMPS), 1,2-Dipalmitoyl-sn-Glycero-3-[Phospho-L-Serine] (DPPS), 1,2-Distearoyl-sn-Glycero-3-[Phospho-L-Serine] (DSPS), 1,2-Dioleoyl-sn-Glycero-3-[Phospho-L-Serine] (DOPS), 1-Palmitoyl-2-Oleoyl-sn-Glycero-3-[Phospho-L-Serine] (POPS).

### EXAMPLE 8

This example illustrates the immunization of mice and rabbits with NspA-liposome formulations.

Groups of female BALB/c mice (Charles River Laboratories, St-Constant, Quebec, Canada) were immunized intramuscularly (IM) three or four times at two-week intervals with 20 μg of recombinant NspA protein adsorbed to 10% aluminium hydroxide adjuvant (Alhydrogel™ 2%: Brenntag Biosector, Denmark), with 20 μg of recombinant NspA incorporated into different liposome preparations or, as control, with protein-free liposome formulations. Blood samples were collected from the orbital sinus prior to each immunization and two weeks after the last injection. The serum samples were stored at −20° C.

New Zealand White female rabbits (2.5 Kg, Charles River) were immunized IM three or four times at three-week intervals at several sites with 100 μg of recombinant NspA protein adsorbed to 10% aluminium hydroxide adjuvant (Alhydrogel™ 2%: Brenntag Biosector, Denmark), with 100 μg of recombinant NspA protein incorporated in different liposome formulations or, as control, with protein-free liposome formulations. Serum samples were collected before each immunization and three weeks after the last injection. The serum samples were stored at −20° C.

### EXAMPLE 9

This example illustrates the analysis by ELISA of mouse and rabbit sera.

The antibody response of immunized animals was determined by enzyme-linked immunosorbent assay (ELISA). Microtiter plates were coated overnight at room temperature with 0.1 ml/well of either purified recombinant NspA at a concentration of 0.5 μg/ml in phosphate buffer (50 mM NaH2PO4, pH 4.3), or OM preparation extracted from the meningococcal strain 608B at a concentration of 0.25 μg of protein per ml in carbonate buffer (15 mM Na2CO3; 35 mM NaHCO3, pH 9.6). Plates were blocked with phosphate-buffered saline (PBS) buffer containing 0.5% (wt/vol) bovine serum albumin (BSA) for 1 h at 37° C. and then incubated for 1 h with serial dilutions of the rabbit and mouse sera. After the incubation period, the plates were washed 3 times with washing buffer (PBS containing 0.02% tween-20). Alkaline phosphatase-conjugated AffiniPure goat anti-mouse IgG+IgM (H+L) or anti-rabbit IgG were diluted in PBS containing 3% (wt/vol) BSA, and 0.1 ml of this solution was added to each well. After an additional incubation of 60 min at 37° C., plates were washed 3 times with washing buffer. One hundred μl of p-nitrophenyl phosphate disodium solution in 10% diethanolamine (pH 9.6) was added to each well. Following incubation for 1 h at room temperature, the OD405 nm was read with a Spectra Max microplate reader (Molecular Devices). The serum dilution for which an absorbance reading of 0.1 (□=410/630 nm) was recorded after background subtraction was considered to be the titer of this serum. All of the antisera raised by immunization with formulations containing the recombinant NspA protein reacted strongly against the recombinant NspA. In addition, as presented in Table 8, all post-immunization sera reacted against meningococcal OMP extracted from strain 608B. These results suggest that a significant proportion of the antibodies induced by immunization do react with native NspA protein when inserted into the meningococcal membranes. Titers below 200 were recorded from sera collected from mice and rabbits immunized with protein-free liposome preparations (Data not shown).

TABLE 8
Analysis of mouse and rabbit antisera collected after
immunization with different NspA-liposome formulations.

| | | Lipid | Reciprocal of anti-OMP | |
|---|---|---|---|---|
| | | concentration | titer of antisera from | |
| | | used to prepare | (standard deviation)²: | |
| Formulations¹ | Method | liposomes | Mouse | Rabbit (s)³ |
| Recombinant NspA⁴ | — | — | 5800 ± 3414 | 409600 (1) |
| N. meningitidis | Dialysis | 75 mM | 64000 ± 25600 | Nd |
| 100% + MPLA | | | | |
| N. lactamica 100% + MPLA | | | 54400 ± 36765 | 102400 (2) |
| E. coli:Chol | | | nd | 409600 (3) |
| (7:2) | | | | |
| E. coli:Chol | | | 70400 ± 38400 | >409600 (4) |
| (7:2) + MPLA | | | | |
| E. coli 100% | | | 208000 ± 175471 | 409600 (5) |
| | | | | >409600 (6) |
| | | | | >409600 (7) |
| | | | | >409600 (8) |
| E. coli 100% | | 6 mM | nd | 204800 (9) |
| | | | | >409600 (10) |
| E. coli:Chol | Rapid | | nd | 204800 (11) |
| (7:2) | dilution | | | 204800 (12) |

¹Mice and rabbits were immunized with recombinant NspA protein or recombinant NspA protein incorporated into different liposome formulations as described in Example 8.
²Sera were tested by ELISA against recombinant NspA and against OMP from N. meningitidis strain 608B. Preimmune sera showed no reactivity against recombinant NspA and against OMP from N. meningitidis strain 608B in ELISA. nd, not determined.
³Number between parentheses indicates the rabbit identification number.
⁴Recombinant NspA protein adsorbed to 10% aluminium hydroxide adjuvant.

### EXAMPLE 10

This example illustrates the accessibility of antibodies raised against NspA-liposome preparations at the surface of N. meningitidis strains.

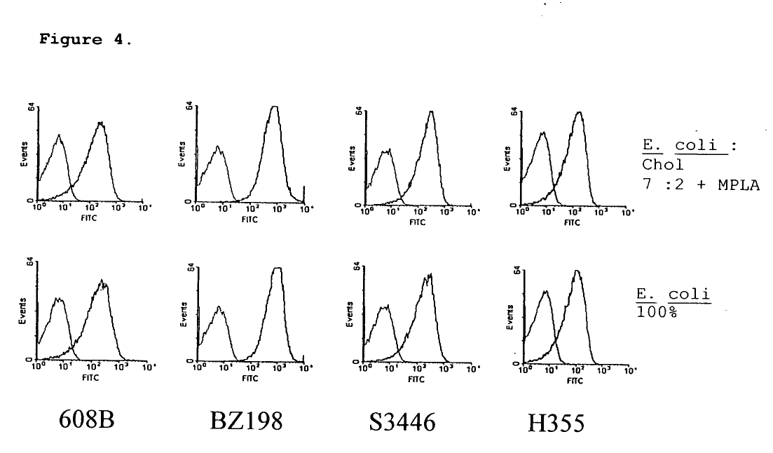

N. meningitidis strains were grown in Mueller-Hinton (MH) broth containing 0.25% dextrose at 37° C. in a 8% CO₂ atmosphere to give an OD_{490 nm} of 0.500 (˜10⁸ CFU/ml). Dilutions of anti-NspA or control sera were then added to the adjusted bacterial culture and incubated for 2 h at 4° C. with agitation. Samples were washed twice in blocking buffer [phosphate-buffered saline (PBS) containing 2% bovine serum albumin (BSA)], and then 1 ml of goat fluorescein (FITC)— conjugated anti-mouse IgG+IgM (H+L) specific or anti-rabbit IgG (H+L) diluted in blocking buffer was added. After an additional incubation period of 60 min. at room temperature with agitation, samples were washed twice in PBS buffer and fixed with 0.3% formaldehyde in PBS buffer for 18 h at 4° C. Cells were kept in the dark at 4° C. until analyzed by flow cytometry (Epics® XL; Beckman Coulter, Inc.). Flow cytometric analysis revealed that antibodies present in the NspA-specific sera from mouse and rabbits immunized with NspA-liposome formulations recognized their corresponding surface exposed epitopes on the meningococcal strain 608B more efficiently than those present in the sera from rabbit immunized with recombinant NspA protein adsorbed to 10% aluminium hydroxide (Table 9). Indeed, binding indexes higher than 25 were recorded for rabbits immunized with recombinant NspA-liposome formulations comparatively to a binding index of 16 recorded for the rabbit immunized with recombinant NspA adsorbed to 10% aluminium hydroxide. It was determined that more than 80% of the 10,000 meningococcal cells analyzed were labelled with the antibodies present in the NspA-specific sera from mouse immunized with different NspA-liposome formulations. In addition, it was also determined that more than 90% of the meningococcal cells analyzed were labelled with the antibodies present in the NspA-specific sera from rabbits immunized with different liposome formulations. FIG. 4 shows that the NspA-specific rabbit antibodies raised after immunization with two different NspA-liposome formulations (E. coli:Chol (7:2)+MPLA; E. coli 100%) can recognized their specific epitopes at the surface of distinct serogroup B meningococcal strains. These observations clearly demonstrate that NspA-specific antibodies present in the sera from mouse and rabbit immunized with NspA-liposome formulations recognize accessible epitopes at the surface of intact meningococcal cells. Antibodies present in the sera collected from mice and rabbits immunized with protein-free liposome preparations did not attach to the meningococcal cells (data not shown).

TABLE 9
Evaluation of the attachment of NspA-specific antibodies at the
surface of intact N. meningitidis strain 608B cells.

| | | Lipid | Antisera from²: | | | | |
|---|---|---|---|---|---|---|---|
| | | concentration | Mouse | | Rabbit(s) | | |
| | | used to | % of | | | % of | |
| | | prepare | labelled | | Rabbit | labelled | |
| Formulations¹ | Method | liposomes | cells³ | BI⁴ | number | cells | BI |
| Recombinant NspA⁵ | — | — | nd | nd | 1 | 78 | 16 |
| N. meningitidis | Dialysis | 75 mM | 80.3 | 30 | — | nd | nd |
| 100% + MPLA | | | | | | | |
| N. lactamica | | | 90.6 | 41 | 2 | 92 | 36.1 |
| 100% + MPLA | | | | | | | |
| E. coli:Chol | | | nd | nd | 3 | 96 | 38.7 |
| (7:2) | | | | | | | |
| E. coli:Chol | | | 87.6 | 20.6 | 4 | 98 | 62.6 |
| (7:2) + MPLA | | | | | | | |
| E. coli 100% | | | nd | nd | 5 | 91 | 33.4 |
| | | | | | 6 | 92 | 41.1 |
| | | | | | 7 | 98 | 98.6 |
| | | | | | 8 | 90 | 25.6 |
| E. coli 100% | | 6 mM | nd | nd | 9 | 98 | 64 |
| | | | | | 10 | 99 | 88 |
| E. coli:Chol | Rapid | | nd | nd | 11 | 98 | 40 |
| (7:2) | dilution | | | | 12 | 96 | 26 |

¹Mice and rabbits were immunized with recombinant NspA-liposome formulations as described in example 8.
²Pooled sera were diluted 1/20 to perform the cytofluorometric assay.
³% of labelled cells out of the 10,000 cells analyzed.
⁴The binding index. (BI) was calculated as the median fluorescence value obtained after labelling the cells with an immune serum divided by the fluorescence value obtained for a control without sera. A fluorescence value of 1 indicated that there was no binding of antibodies at the surface of intact meningococcal cells. nd, not determined.
⁵Recombinant NspA protein adsorbed to 10% aluminum hydroxide adjuvant.

### EXAMPLE 11

This example illustrates the bactericidal activities of anti-NspA antibodies present in mouse and rabbit sera.

Bacteria were plated on chocolate agar plate and incubated at 37° C. in a 8% CO₂ atmosphere for 16 h or were grown in Mueller-Hinton (MH) broth containing 0.25% dextrose at 37° C. in a 8% CO₂ atmosphere to give an OD₆₂₀ nm of 0.600. After the incubation period, bacteria were suspended in bacteriolysis buffer [Hanks' Balanced Salt Solution (HBSS) and 1% hydrolyzed casein, pH 7.3] to an OD₄₉₀ₙₘ of 0.300 and diluted to 8×10⁴ CFU/ml. The bactericidal assay was performed by mixing 25 μl of the bacterial suspension with 50 μl of diluted heat-inactivated test serum. This suspension was incubated for 15 min at 37° C., 8% CO₂ with agitation (225 rpm). The rabbit or human serum as a source of complement was then added to a final concentration of 25%, and the mixture was incubated for an additional 60 min at 37° C., 8% CO₂ with agitation (225 rpm). At the end of the incubation period, the number of viable bacteria was determined by plating 10 μl of the assay mixture on chocolate agar plate. The plates were incubated at 37° C. in an 8% CO₂ atmosphere for 18-24 h. The control consisted of bacteria incubated with heat-inactivated sera collected from mice before immunization and rabbit complement. The % of lysis was determined using the following mathematical formula:

$$100 - [ \frac{\begin{matrix}CFU obtained when the bacteria \\ were incubated with immune sera\end{matrix}}{CFU obtained with pre \text{-} bleed sera} \times 100 ]$$

Bactericidal antibodies were found to be present in the sera collected from mice and rabbit immunized with the purified recombinant NspA protein incorporated in liposome (Table 10).

Importantly, bactericidal antibodies were not present in the sera collected from rabbit immunized with recombinant NspA protein adsorbed to 10% aluminium hydroxide. In addition, sera collected from rabbits immunized with two different liposome formulations (E. coli: Chol (7:2)+MPLA, E. coli 100%) were also found to be bactericidal against three distinct serogroup B strains (Table 11). This latter result indicates that immunization with NspA-liposome formulations can induce the production of cross-bactericidal antibodies. These data demonstrate that incorporation of purified recombinant NspA protein into liposome considerably enhanced the immune response against the native protein.

TABLE 10
Bactericidal activity of antisera raised against NspA-
liposome formulations against the meningococcal strain 608B.

| | | Lipid | | |
|---|---|---|---|---|
| | | concentration | | |
| | | used to | % of lysis | |
| | | prepare | Antisera from²: | |
| Formulations¹ | Method | liposomes | Mouse | Rabbit(s)³ |
| Recombinant | — | — | nd | 19 (1) |
| NspA⁴ | | | | |
| N. meningitidis | Dialysis | 75 mM | 79 | nd |
| 100% + MPLA | | | | |
| N. lactamica | | | 84 | 94 (2) |
| 100% + MPLA | | | | |
| E. coli:Chol | | | nd | 93 (3) |
| (7:2) | | | | |
| E. coli:Chol | | | 79 | 95.7 (4) |
| (7:2) + MPLA | | | | |
| E. coli 100% | | | 85 | 86.4 (5) |
| | | | | 89 (6) |
| | | | | 100 (7) |
| | | | | 75 (8) |
| E. coli 100% | | 6 mM | nd | 77 (9) |
| | | | | 99 (10) |
| E. coli:Chol | Rapid | | nd | 75 (11) |
| (7:2) | dilution | | | 80 (12) |

¹Mice and rabbits were immunized with recombinant NspA-liposome formulations as described in example 8.
²Antisera raised against recombinant NspA preparations were tested for their ability to induce complement-mediated killing of the meningococcal strain 608B. Sera were diluted 1/10.
nd, not determined.
³Number between parentheses indicates the rabbit number.
⁵Recombinant NspA protein adsorbed to 10% aluminium hydroxide adjuvant.

TABLE 11
Bactericidal activity of rabbit antisera collected
after immunization with different NspA-liposome formulations.

| Formulations | % of lysis against strains¹: | | | |
|---|---|---|---|---|
| (rabbit | 608B | BZ198 | S3446 | H355 |
| number) | (B:2a:P1.2) | (B:NT:P-) | (B:14:P1.23, 14) | (B:15:P1.15) |
| E. coli:Chol | 95.7 | 100 | 97.2 | 91.6 |
| (7:2) + | | | | |
| MPLA (4) | | | | |
| E. coli 100% | 86.4 | 99.8 | 65.0 | 62.2 |
| (5) | | | | |

¹Rabbit sera raised against recombinant NspA preparations were tested for their ability to induce complement-mediated killing of the four meningococcal strains. Sera were diluted 1/10.

### EXAMPLE 12

This example illustrates the incorporation of recombinant NspA into different liposome formulations.

Purified recombinant NspA protein (rNspA) was first precipitated by addition of absolute ethanol (vol/vol). The precipitated rNspA was solubilized in 1 ml of PBS buffer containing 1% (wt/vol) of sodium dodecyl sulfate (SDS; Sigma chemical), and heated at 100° C. for 10 min. The rNspA solution was diluted with 1 ml of PBS buffer containing 15% (wt/vol) of n-octyl β-D-glucopyranoside (OG, Sigma) and incubated at room temperature for 3 h.

Liposomes made of 1,2-Dimyristoyl-sn-Glycero-3-Phosphocholine (DMPC; Avanti polar lipids, Alabaster, Ala.), 1,2-Dimyristoyl-sn-Glycero-3-[Phospho-L-Serine] (DMPS, Avanti), and cholesterol (Chol; Avanti), and liposomes made of DMPC, 1,2-Dimyristoyl-3-Trimethylammonium-Propane (DMTAP, Avanti), and cholesterol were prepared using a dialysis method (Muttilainen et al. 1995, Microb Pathog., 18:423-36). Briefly, lipids were dissolved in a chloroform:methanol solution (2:1) in a round bottom glass flask and dried using a rotatory evaporator (Rotavapor, Büchi, Switzerland) to achieve an even film on the vessel. The rNspA protein-detergent solution was added to the lipid film and mixed gently until the film was suspended. The mixture was slightly opalescent in appearance. The mixture was then extensively dialysed against PBS buffer (pH 7.4) to remove detergent and induce liposome formation. After dialysis, the resulting milky suspension was sequentially extruded through 1000, 400, 200, and 100 nm polycarbonate filters using a stainless steel extrusion device (Lipex Biomembranes, Vancouver, Canada). The rNspA not incorporated into the liposome was removed by centrifugation at 20000×g for 15 min at 4° C. The liposome solution was centrifuged at 250000×g for 1 h at 4° C. and the pellet was suspended with PBS buffer containing 0.3 M of sucrose. Vesicle size and homogeneity were evaluated by quasi-elastic light scattering with a submicron particles analyzer (model N4 Plus, Beckman Coulter). Using this apparatus, it was estimated that the liposome size in the different preparations was approximately 150 nm. All liposome preparations were sterilized by filtration through a 0.22 μm membrane and stored at +4° C. or −80° C. until used. The amount of recombinant protein incorporated in the liposome was estimated by MicroBCA (Pierce, Rockford, Ill.) after protein extraction of rNspA-liposome preparations with chloroform:methanol solution (2:1) as described by Wessel and Flugge (Anal. Biochem. 1984, 138:141-143).

A method based on diafiltration was used as alternate method to generate the rNspA-liposome formulations. For this method, lipids were suspended in 8% OG at 50° C. One volume of rNspA protein prepared as described above was combined with different volumes of lipid suspension and incubated 15 minutes at 37° C.

The lipid/protein mixture was diluted into HEPES buffer saline (HBS) to induce the formation of liposome vesicles. The resulting suspension was passed through two stacked 100 nm polycarbonate filters using a stainless steel extrusion device (Lipex Biomembranes, Vancouver, Canada). The liposome formulations were ultrafiltered to the desired final volume, then diafiltered against 10 volumes of HBS to remove free protein and detergent using a 500,000 nominal molecular weight cutoff cartridge from A/G Technology Corp. Finally, preparations were sterilized through a 0.22 μm membrane and vesicle size and homogeneity were evaluated as described above. All preparations were stored at +4° C. until used.

### EXAMPLE 13

This example illustrates the immunization of mice with rNspA-liposome formulations.

Groups of female BALB/c mice (4 to 6 weeks old; Charles River Laboratories, St-Constant, Quebec, Canada) were immunized intramuscularly (IM) four times at three-week intervals with 20 μg of rNspA protein adsorbed to 10% aluminium hydroxide adjuvant (Alhydrogel™ 2%: Brenntag Biosector, Denmark), or with 20 μg of rNspA incorporated into different liposome preparations. Blood samples were collected from the orbital sinus prior to each immunization and three weeks after the last injection. The serum samples were stored at −20° C.

### EXAMPLE 14

This example illustrates the immunization of rabbits with rNspA-liposome formulations.

New Zealand White female rabbits (2.5 Kg, Charles River) were immunized IM four times at three-week intervals at several sites with 100 μg of rNspA protein adsorbed to 10% aluminium hydroxide adjuvant (Alhydrogel™ 2%: Brenntag Biosector, Denmark) or with 100 μg of rNspA protein incorporated in different liposome formulations. Serum samples were collected before each immunization and three weeks after the last injection. The serum samples were stored at −20° C.

### EXAMPLE 15

This example illustrates the analysis by ELISA of sera from rabbits immunized with rNspA-liposome formulations.

The antibody response of immunized animals was determined by enzyme-linked immunosorbent assay (ELISA). Microtiter plates were coated overnight at room temperature with 0.1 ml/well of either rNspA at a concentration of 0.5 μg/ml in phosphate buffer (50 mM NaH₂PO₄, pH 4.2), or OM preparation extracted from the meningococcal strain 608B at a concentration of 2.5 μg of protein per ml in carbonate buffer (15 mM Na₂CO₃; 35 mM NaHCO₃, pH 9.6). Plates were blocked with phosphate-buffered saline (PBS) buffer containing 0.5% (wt/vol) bovine serum albumin (BSA) for 30 minutes at 37° C. and then incubated for 1 h with serial dilutions of the rabbit sera. After the incubation period, the plates were washed 3 times with washing buffer (PBS containing 0.02% tween-20). Alkaline phosphatase-conjugated AffiniPure goat anti-rabbit IgG were diluted in PBS containing 3% (wt/vol) BSA, and 0.1 ml of this solution was added to each well. After an additional incubation of 60 min at 37° C., plates were washed 3 times with washing buffer. One hundred μl of p-nitrophenyl phosphate disodium solution in 10% diethanolamine (pH 9.6) was added to each well. Following incubation for 1 h at room temperature, the OD_{405 nm} was evaluated using a Spectra Max microplate reader (Molecular Devices). The serum dilution for which an absorbance reading corresponding to 2 times the OD value obtained for the preimmune serum (λ=405/630 nm) was considered to be the titer of this serum. All sera collected from rabbits immunized with formulations containing the rNspA protein reacted strongly with the rNspA as evaluated by ELISA (data not shown). As presented in Table 1, stronger titers against meningococcal OMP were determined for sera collected from rabbits immunized with rNspA-liposome (317979±133703; means±standard deviation) comparatively to the titers obtained for rabbits immunized with rNspA adsorbed to Alum adjuvant (21333±7390). These results suggest that a significant proportion of the antibodies induced by immunization do react with native NspA protein when inserted into the meningococcal membranes.

TABLE 12
Analysis of sera from rabbits immunized with different rNspA-liposome
formulations by ELISA, cytofluorometry and bactericidal assay (SBA).

| | Lipid | | | Surface binding | | |
|---|---|---|---|---|---|---|
| | concentration | | | assay³ | | |
| | used to | | Reciprocal | | % of | SBA⁶ |
| | prepare | Rabbit | of anti- | | labelled | % of |
| Formulations¹ | liposomes | number | OMP titer² | BI⁴ | cells⁵ | lysis |
| rNspA⁷ | — | 1 | 25600 | 29 | 97 | 16 |
| | | 2 | 25600 | 25 | 97 | 23 |
| | | 3 | 12800 | 8 | 79 | 13 |
| DMPC:DMPS:Chol | 75 mM | 4 | >409600 | 53 | 99 | 54 |
| (4:1:1) | | 5 | 409600 | 169 | 100 | 100 |
| | | 6 | >409600 | 154 | 100 | 100 |
| | | 7 | 102400 | 39 | 97 | 38 |
| | | 8 | 409600 | 81 | 100 | 72 |
| | | 9 | 409600 | 94 | 100 | 70 |
| | | 10 | 409600 | 23 | 93 | 17 |
| | | 11 | >409600 | 91 | 99 | 81 |
| | | 12 | >409600 | 81 | 100 | 77 |
| | | 13 | 409600 | 27 | 95 | 20 |
| | | 14 | >409600 | 47 | 98 | 41 |
| DMPC:DMTAP:Chol | | 15 | >409600 | 230 | 100 | 100 |
| (4:1:1) | | 16 | 204800 | 33 | 98 | 39 |
| | | 17 | >409600 | 121 | 100 | 79 |
| | | 18 | 204800 | 132 | 100 | 55 |
| | | 19 | 409600 | 97 | 99 | 55 |
| | | 20 | 409600 | 153 | 100 | 87 |
| | | 21 | 409600 | 127 | 100 | 89 |
| | | 22 | >409600 | 269 | 100 | 100 |
| | | 23 | 409600 | 132 | 100 | 71 |
| | | 24 | 204800 | 145 | 100 | 79 |
| | | 25 | >409600 | 213 | 100 | 94 |
| DMPC:DMPS:Chol | 30 mM | 26 | 102400 | 36 | 97 | 42 |
| (4:1:1) | | 27 | 409600 | 59 | 99 | 80 |
| | | 28 | 102400 | 11 | 79 | 19 |
| | | 29 | 102400 | 40 | 98 | 42 |
| | | 30 | 204800 | 22 | 88 | 18 |
| | | 31 | 409600 | 70 | 100 | 68 |
| | | 32 | 102400 | 71 | 100 | 61 |
| | | 33 | >409600 | 49 | 99 | 54 |
| DMPC:DMTAP:Chol | | 34 | 51200 | 31 | 96 | 22 |
| (4:1:1) | | 35 | >409600 | 121 | 100 | 52 |
| | | 36 | >409600 | 169 | 100 | 83 |
| | | 37 | 409600 | 147 | 100 | 78 |
| | | 38 | 204800 | 131 | 100 | 56 |
| | | 39 | >409600 | 216 | 100 | 96 |
| | | 40 | 51200 | 112 | 100 | 61 |
| | | 41 | 204800 | 110 | 99 | 52 |

¹Rabbits were immunized intramuscularly four times with 100 μg of rNspA protein adsorbed to 10% aluminium hydroxide adjuvant, or with 100 μg of rNspA incorporated into different liposome preparations as described in Example 12.
²Sera were tested by ELISA against OMP extracted from N. meningitidis strain 608B. Preimmune sera showed no reactivity against the meningococcal OMP preparation as evaluated by ELISA.
³Sera were diluted 1/20 to perform the cytofluorometric assay as described in Example 16.
⁴The binding index (BI) was calculated as the median fluorescence value obtained after labelling the cells with an immune serum divided by the fluorescence value obtained for a control without sera. A fluorescence value of 1 indicated that there was no binding of antibodies at the surface of intact meningococcal cells.
⁵% of labelled cells out of the 10,000 cells analyzed.
⁶Antisera raised against rNspA preparations were tested for their ability to induce complement-mediated killing of the meningococcal strain 608B as described in Example 17. Sera were diluted 1/40.
⁷rNspA protein was adsorbed to 10% aluminium hydroxide adjuvant.

### EXAMPLE 16

This example illustrates the accessibility of antibodies raised against NspA-liposome formulations at the surface of N. meningitidis cells.

N. meningitidis strains were grown in Mueller-Hinton (MH) broth containing 0.25% dextrose at 37° C. with agitation (225 rpm) in a 8% CO₂ atmosphere to give an OD₄₉₀ nm of 0.500 (˜10⁸ CFU/ml). Dilutions of anti-NspA or control sera were then added to the adjusted bacterial culture and incubated for 2 h at 4° C. with agitation. Samples were washed twice with blocking buffer [phosphate-buffered saline (PBS) containing 2% bovine serum albumin (BSA)], and then 1 ml of goat fluorescein (FITC)-conjugated anti-rabbit IgG (H+L) diluted in blocking buffer was added. After an additional incubation period of 60 min at room temperature with agitation, samples were washed twice in PBS buffer and fixed with 0.3% formaldehyde in PBS buffer for 18 h at 4° C. Cells were kept in the dark at 4° C. until their analysis by flow cytometry (Epics® XL; Beckman Coulter, Inc.). Binding index (BI) was calculated as the median fluorescence value obtained after labelling the cells with an immune serum divided by the fluorescence value obtained for a control without sera. A fluorescence value of 1 indicated that there was no binding of antibodies at the surface of intact meningococcal cells. Flow cytometric analysis revealed that antibodies present in sera from rabbits immunized with rNspA-liposome formulations recognized their corresponding surface exposed epitopes on the meningococcal cells more efficiently than those present in the sera from rabbits immunized with rNspA protein adsorbed to 10% aluminium hydroxide (Table 12). Indeed, binding indexes recorded for rabbits immunized with rNspA-liposome formulations were generally higher (11≦BI≦269) than the binding indexes recorded for the rabbit immunized with rNspA adsorbed to 10% aluminium hydroxide (BI≦29). These observations clearly demonstrate that NspA-specific antibodies present in the sera from rabbits immunized with rNspA-liposome formulations recognize accessible epitopes at the surface of intact meningococcal cells.

### EXAMPLE 17

This example illustrates the bactericidal activities of anti-NspA antibodies present in rabbit sera.

Bacteria were plated on BHI agar plate containing 1% horse serum (Gibco BRL) and incubated at 37° C. in a 8% CO₂ atmosphere for 16 h. Mueller-Hinton (MH) broth containing 0.25% dextrose was inoculated with bacteria from BHI agar plate and was incubated with agitation (225 rpm) at 37° C. in a 8% CO₂ atmosphere until to obtain an OD_{620 nm} of 0.600. After the incubation period, bacteria were suspended in bacteriolysis buffer [Hanks' Balanced Salt Solution (HBSS) and 0.1% gelatin, pH 7.2] to an OD_{490 nm} of 0.300 and diluted to 8×10⁴ CFU/ml. The bactericidal assay was performed by mixing 25 μl of the adjusted bacterial suspension with 50 μl of diluted heat-inactivated rabbit serum. As source of complement, a volume of 25 μl (25% v/v) of normal human serum selected for its weak specific killing activity for meningococcal cell was added, and the mixture was incubated for 60 min at 37° C., 8% CO₂ with agitation (225 rpm). At the end of the incubation period, the number of viable bacteria was determined by plating 10 μl of the assay mixture on chocolate agar plate. The plates were incubated at 37° C. in an 8% CO₂ atmosphere for 18-24 h. The control consisted of bacteria incubated with heat-inactivated sera collected from rabbits before immunization and human complement. The % of lysis was determined using the following mathematical formula:

$$100 - [ \frac{\begin{matrix}CFU obtained when the bacteria \\ were incubated with immune sera\end{matrix}}{CFU obtained with pre \text{-} bleed sera} \times 100 ]$$

Bactericidal antibodies were found to be present in most sera collected from rabbits immunized with the rNspA protein incorporated in liposome (Table 12). Importantly, bactericidal antibodies were not present in the sera collected from rabbit immunized with rNspA protein adsorbed to 10% aluminium hydroxide. In addition, sera collected from rabbits immunized with two different liposome formulations (DMPC:DMPS:Chol, DMPC:DMTAP:Chol; 75 mM) were also found to be bactericidal against three distinct serogroup B strains and one serogroup A strain (Z4063) (Table 13). This latter result indicates that immunization with rNspA-liposome formulations can induce the production of cross-bactericidal antibodies. These data demonstrate that incorporation of rNspA protein into liposome considerably enhanced the functional immune response against the native protein.

TABLE 13
Bactericidal activity of rabbit antisera collected after immunization
with different rNspA-liposome formulations.

| | | % of lysis against strains¹: | | | |
|---|---|---|---|---|---|
| | Rabbit | 608B | SWZ107 | H355 | Z4063 |
| Formulations | number | (B:2a:P1.2) | (B:4:P1.2) | (B:15:P1.15) | (A:4:P1.7) |
| DMPC:DMPS:Chol | 4 | 85 | 73 | 75 | 98 |
| (4:1:1) 75 mM | 5 | 100 | 100 | 100 | 100 |
| | 6 | 100 | 100 | 99 | 100 |
| DMPC:DMTAP:Chol | 15 | 100 | 100 | 100 | 100 |
| (4:1:1) 75 mM | 17 | 96 | 98 | 90 | 100 |

¹Rabbit sera raised against rNspA preparations were tested for their ability to induce complement-mediated killing of the four meningococcal strains. Sera were diluted 1/20.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated. The entire disclosures of all applications, patents and publications, cited herein and of corresponding U.S. Provisional Application Ser. No. 60/658,815, filed Mar. 7, 2005 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A pharmaceutical composition comprising a liposome associated with at least one polypeptide comprising SEQ ID No: 2 or a fragment or analog thereof.

2. A pharmaceutical composition according to claim 1, wherein said composition comprises a liposome associated with at least one polypeptide comprising SEQ ID No: 2.

3. A pharmaceutical composition according to claim 1, wherein said composition comprises a liposome associated with at least one polypeptide consisting of SEQ ID No: 2 or a fragment or analog thereof.

4. A pharmaceutical composition according to claim 1, wherein said composition comprises a liposome associated with at least one polypeptide consisting of SEQ ID No: 2.

5. A pharmaceutical composition comprising a liposome associated with at least one epitope bearing portion of a polypeptide comprising SEQ ID No: 2 or a fragment or analog thereof.

6. A pharmaceutical composition according to claim 5, wherein said composition comprises a liposome associated with at least one epitope bearing portion of a polypeptide comprising SEQ ID No: 2.

7. A pharmaceutical composition comprising a liposome associated with at least one isolated polypeptide, wherein said isolated polypeptide is selected from:
(a) a polypeptide having at least 70% identity to a second polypeptide comprising SEQ ID No: 2 or fragment or analog thereof;
(b) a polypeptide having at least 80% identity to a second polypeptide comprising SEQ ID No: 2 or a fragment or analog thereof;
(c) a polypeptide having at least 95% identity to a second polypeptide comprising SEQ ID No: 2 or a fragments or analog thereof;
(d) a polypeptide comprising SEQ ID No: 2 or a fragment or analog thereof;
(e) a polypeptide capable of raising antibodies having binding specificity for a polypeptide comprising SEQ ID No: 2 or a fragment or analog thereof;
(f) an epitope bearing portion of a polypeptide comprising SEQ ID No: 2 or a fragment or analog thereof;
(g) the polypeptide of (a), (b), (c), (d), (e) or (f) wherein the N-terminal Met residue is deleted; and
(h) the polypeptide of (a), (b), (c), (d), (e), (f) or (g) wherein the secretory amino acid sequence is deleted.

8. A pharmaceutical composition according to claim 7, wherein said isolated polypeptide is selected from:
(a) a polypeptide having at least 70% identity to a second polypeptide comprising SEQ ID No: 2;
(b) a polypeptide having at least 80% identity to a second polypeptide comprising SEQ ID No: 2;
(c) a polypeptide having at least 95% identity to a second polypeptide comprising SEQ ID No: 2;
(d) a polypeptide comprising SEQ ID No: 2;
(e) a polypeptide capable of raising antibodies having binding specificity for a polypeptide comprising SEQ ID No: 2;
(f) an epitope bearing portion of a polypeptide comprising SEQ ID No: 2;
(g) the polypeptide of (a), (b), (c), (d), (e) or (f) wherein the N-terminal Met residue is deleted; and
(h) the polypeptide of (a), (b), (c), (d), (e), (f) or (g) wherein the secretory amino acid sequence is deleted.

9. A pharmaceutical composition comprising a liposome associated with at least one isolated polynucleotide, wherein said isolated polynucleotide is selected from:
(a) a polynucleotide encoding a polypeptide having at least 70% identity to a second polypeptide comprising SEQ ID No: 2 or a fragment or analog thereof;
(b) a polynucleotide encoding a polypeptide having at least 80% identity to a second polypeptide comprising SEQ ID No: 2 or a fragment or analog thereof;
(c) a polynucleotide encoding a polypeptide having at least 95% identity to a second polypeptide comprising SEQ ID No: 2 or a fragment or analog thereof;
(d) a polynucleotide encoding a polypeptide comprising SEQ ID No: 2 or a fragment or analog thereof;
(e) a polynucleotide encoding a polypeptide capable of raising antibodies having binding specificity for a polypeptide comprising SEQ ID No: 2 or a fragment or analog thereof;
(f) a polynucleotide encoding an epitope bearing portion of a polypeptide comprising SEQ ID No: 2 or a fragment or analog thereof;
(g) a polynucleotide comprising SEQ ID No: 1 or a fragment or analog thereof; and
(h) a polynucleotide that is complementary to a polynucleotide in (a), (b), (c), (d), (e), (f) or (g).

10. A pharmaceutical composition according to claim 9, wherein said isolated polynucleotide is selected from:
(a) a polynucleotide encoding a polypeptide having at least 70% identity to a second polypeptide comprising SEQ ID No: 2;
(b) a polynucleotide encoding a polypeptide having at least 80% identity to a second polypeptide comprising SEQ ID No: 2;
(c) a polynucleotide encoding a polypeptide having at least 95% identity to a second polypeptide comprising SEQ ID No: 2;
(d) a polynucleotide encoding a polypeptide comprising SEQ ID No: 2;
(e) a polynucleotide encoding a polypeptide capable of raising antibodies having binding specificity for a polypeptide comprising SEQ ID No: 2;
(f) a polynucleotide encoding an epitope bearing portion of a polypeptide comprising SEQ ID No: 2;
(g) a polynucleotide comprising SEQ ID No: 1 or fragments or analogs thereof; and
(h) a polynucleotide that is complementary to a polynucleotide in (a), (b), (c), (d), (e), (f) or (g).

11. A pharmaceutical comprising a liposome associated with chimeric polypeptides comprising two or more polypeptides comprising SEQ ID No: 2 or a fragment or analog thereof, wherein said polypeptides are linked as to formed a chimeric polypeptide.

12. A pharmaceutical composition according to claim 10, wherein said composition comprises a liposome associated with chimeric polypeptides comprising two or more polypeptides comprising SEQ ID No: 2 wherein said polypeptides are linked as to form a chimeric polypeptide.

13. A pharmaceutical composition according to claim 1, wherein said liposome comprises lipids selected from synthetic phospholipids, bacterial phospholipids and/or cholesterol.

14. A pharmaceutical composition according to claim 13, wherein said liposome comprises bacterial lipids extracted from E. coli, N. meningitidis, or N. lactamica.

15. A pharmaceutical composition according to claim 1, wherein said liposome comprises lipids selected from phosphatidyl ethers and esters, glycerides, gangliosides, sphyngomyelin, and steroids.

16. A pharmaceutical composition according to claim 13, wherein said lipids are selected from:
1,2-Dilauroyl-sn-Glycero-3-Phosphate (DLPA),
Dimyristoyl-sn-Glycero-3-Phosphate (DMPA),
1,2-Dipalmitoyl-sn-Glycero-3-Phosphate (DPPA),
1,2-Distearoyl-sn-Glycero-3-Phosphate (DSPA),
1,2-Dioleoyl-sn-Glycero-3-Phosphate (DOPA),
1-Palmitoyl-2-Oleoyl-sn-Glycero-3-Phosphate (POPA),
1,2-Dilauroyl-sn-Glycero-3-Phosphocholine (DLPC),
1,2-Ditridecanoyl-sn-Glycero-3-Phosphocholine,
1,2-Dimyristoyl-sn-Glycero-3-Phosphocholine (DMPC),
1,2-Dipentadecanoyl-sn-Glycero-3-Phosphocholine,
1,2-Dipalmitoyl-sn-Glycero-3-Phosphocholine (DPPC),
1,2-Diheptadecanoyl-sn-Glycero-3-Phosphocholine,
1,2-Distearoyl-sn-Glycero-3-Phosphocholine (DSPC),
1,2-Dimyristoleoyl-sn-Glycero-3-Phosphocholine,
1,2-Dipalmitoleoyl-sn-Glycero-3-Phosphocholine,
1,2-Dioleoyl-sn-Glycero-3-Phosphocholine (DOPC),
1-Myristoyl-2-Palmitoyl-sn-Glycero-3-Phosphocholine,
1-Myristoyl-2-Stearoyl-sn-Glycero-3-Phosphocholine,
1-Palmitoyl-2-Myristoyl-sn-Glycero-3-Phosphocholine,
1-Palmitoyl-2-Stearoyl-sn-Glycero-3-Phosphocholine,
1-Palmitoyl-2-Oleoyl-sn-Glycero-3-Phosphocholine (POPC),
1-Palmitoyl-2-Linoleoyl-sn-Glycero-3-Phosphocholine,
1,2-Dilauroyl-sn-Glycero-3-Phosphoethanolamine (DLPE),
1,2-Dimyristoyl-sn-Glycero-3-Phosphoethanolamine (DMPE),
1,2-Dipalmitoyl-sn-Glycero-3-Phosphoethanolamine (DPPE),
1,2-Dipalmitoleoyl-sn-Glycero-3-Phosphoethanolamine,
1,2-Distearoyl-sn-Glycero-3-Phosphoethanolamine (DSPE),
1,2-Dioleoyl-sn-Glycero-3-Phosphoethanolamine (DOPE),
1-Palmitoyl-2-Oleoyl-sn-Glycero-3-Phosphoethanolamine (POPE),
1,2-Dilauroyl-sn-Glycero-3-[Phospho-RAC-(1-glycerol)] (DLPG),
1,2-Dimyristoyl-sn-Glycero-3-[Phospho-RAC-(1-glycerol)] (DMPG), 1,2-Dipalmitoyl-sn-Glycero-3-[Phospho-RAC-(1-glycerol)] (DPPG), 1,2-Distearoyl-sn-Glycero-3-[Phospho-RAC-(1-glycerol)] (DSPG),
1,2-Dioleoyl-sn-Glycero-3-[Phospho-RAC-(1-glycerol)] (DOPG),
1-Palmitoyl-2-Oleoyl-sn-Glycero-3-[Phospho-RAC-(1-glycerol)] (POPG),
1,2-Dilauroyl-sn-Glycero-3-[Phospho-L-Serine] (DLPS),
1,2-Dimyristoyl-sn-Glycero-3-[Phospho-L-Serine] (DMPS),
1,2-Dipalmitoyl-sn-Glycero-3-[Phospho-L-Serine] (DPPS),
1,2-Distearoyl-sn-Glycero-3-[Phospho-L-Serine] (DSPS),
1,2-Dioleoyl-sn-Glycero-3-[Phospho-L-Serine] (DOPS), and
1-Palmitoyl-2-Oleoyl-sn-Glycero-3-[Phospho-L-Serine] (POPS).

17. A pharmaceutical composition according to claim 16, wherein said lipids are selected from:
1,2-Dimyristoyl-sn-Glycero-3-Phosphocholine (DMPC),
1,2-Dimyristoyl-sn-Glycero-3-[Phospho-L-Serine] (DMPS), and
1,2-Dimyristoyl-3-Trimethylammonium-Propane (DMTAP).

18. A pharmaceutical composition according to claim 13, wherein said liposome further comprises at least one adjuvant selected from Lipid A, monophosphoryl lipid A (MPLA), lipopolysaccharides, and cytokines.

19. A pharmaceutical composition according to claim 13, wherein said liposome comprises 0 to 25 mol % cholesterol.

20. A pharmaceutical composition according to claim 1, wherein said composition further comprises a pharmaceutically acceptable adjuvant.

21. A method for inducing an immune response against N. meningitidis, in a host, comprising administering to said host an immunogenically effective amount of a pharmaceutical composition according to claim 1 to elicit an immune response.

22. A method for preventing and/or treating a N. meningitidis infection comprising administering to a host in need thereof a prophylactic or therapeutic amount of a pharmaceutical composition according to claim 1.

23. A method for preventing and/or treating a neisserial infection selected from N. meningitidis, N. gonorrhoeae, N. lactamica and N. polysaccharea comprising administering to a host in need thereof a prophylactic or therapeutic amount of a pharmaceutical composition according to claim 1.

24. A method for the treatment or prophylaxis of meningitidis and meningoccemia, in a host, comprising administering to said host an effective amount of a pharmaceutical composition according to claim 1.

25. A method according to claim 20, wherein said host is a mammal.

26. A method according to claim 24, wherein said host is a human.

27. A method according to claim 25, wherein said host is an adult human.

28. A method according to claim 20 wherein said are administered in unit dosage form of about 0.001 to 100 μg/kg (antigen/body weight) with an interval of about 1 to 6 week intervals between immunizations.

29. A diagnostic method for detecting N. meningitidis organism in a biological sample, comprising:
a) obtaining a biological sample from a host;
b) incubating an antibody or fragment thereof reactive with a pharmaceutical composition according to claim 1 with the biological sample to form a mixture; and
c) detecting specifically bound antibody or bound fragment in the mixture which indicates the presence of N. meningitidis.

30. A diagnostic method for detecting N. meningitidis organism in a biological sample, comprising:
a) obtaining a biological sample from a host;
b) incubating a pharmaceutical composition according to claim 1 with the biological sample to form a mixture; and
c) detecting specifically bound antigen or bound fragment in the mixture which indicates the presence of antibody specific to N. meningitidis.

31. A diagnostic method for detecting N. meningitidis organism in a biological sample, comprising:
a) obtaining the biological sample from a host;
b) incubating one or more DNA probes having a DNA sequence encoding a polypeptide comprising SEQ ID No: 2 or a fragment thereof with the biological sample to form a mixture; and
c) detecting specifically bound DNA probe in the mixture which indicates the presence of N. meningitidis bacteria.

32. A diagnostic method for detecting N. meningitidis in a host comprising:
a) labelling an antibody reactive with a pharmaceutical composition according to claim 1 with a detectable label;
b) administering the labelled antibody to the host; and
c) detecting specifically bound labelled antibody or labelled fragment in the host which indicates the presence of N. meningitidis.

33. Use of a pharmaceutical method according to claim 1 for the prophylactic or therapeutic treatment of N. meningitidis infection in an individual susceptible to N. meningitidis infection comprising administering to said individual a therapeutic or prophylactic amount of said.

34. A kit comprising a according to claim 1 for detection of diagnosis of N. meningitidis infection.


SEQUENCE LISTING (32 sequences)

SEQ ID NO: 1 - DNA, 525 bp, N. meningitidis strain 608B
atgaaaaaagcacttgccacactgattgccctcgctctcccggccgccgcactggcggaaggcgcatccggcttttacgtccaagccgatgccgcacacgcaaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgccatttacgacttcgacacccaatcgcccgtcaaaccgtatctcggcgcgcgcttgagcctcaaccgcgcctccgtcgacttgggcggcagcgacagcttcagccaaacctccatcggcctcggcgtattgacgggcgtaagctatgccgttaccccgaatgtcgatttggatgccggctaccgctacaactacatcggcaaagtcaacactgtcaaaaacgtccgttccggcgaactgtccgtcggcgtgcgcgtcaaattctga

SEQ ID NO: 2 - protein, 174 aa, N. meningitidis strain 608B
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSVGVRVKF

SEQ ID NO: 3 - DNA, 30 bp, Artificial
  primer
ggaattccatatgaaaaaagcacttgccac

SEQ ID NO: 4 - DNA, 34 bp, Artificial
  primer
ataagaatgcggccgctcagaatttgacgcgcac

SEQ ID NO: 5 - DNA, 30 bp, Artificial
  primer
tcgaggtacccgtgtaatcgacggcgaagc

SEQ ID NO: 6 - DNA, 28 bp, Artificial
  primer
tcgaggtaccctttacagcatcggcgcg

SEQ ID NO: 7 - DNA, 33 bp, Artificial
  primer
tcgaggtacctgtttttgcgtgtgcggcatcgg

SEQ ID NO: 8 - DNA, 28 bp, Artificial
  primer
tcgaggtaccaaaggcttcagcccgcgc

SEQ ID NO: 9 - DNA, 30 bp, Artificial
  primer
atatgggcccggcgcggttgaggctcaagc

SEQ ID NO: 10 - DNA, 35 bp, Artificial
  primer
atatgggccctccaacacctccatcggcctcggcg

SEQ ID NO: 11 - DNA, 45 bp, Artificial
  primer
cgataatggcgaactgtccgtcggcgtgcgcgtcaaattctgagc

SEQ ID NO: 12 - DNA, 53 bp, Artificial
  primer
ggccgctcagaatttgacgcgcacgccgacggacagttcgccattatcgggcc

SEQ ID NO: 13 - DNA, 31 bp, Artificial
  primer
atatgggcccgtagttgtagcggtagccggc

SEQ ID NO: 14 - DNA, 31 bp, Artificial
  primer
tcgaggtacccgtgtaatcgacggcgaagcg

SEQ ID NO: 15 - DNA, 31 bp, Artificial
  primer
tcgaggtaccctttacagcatcggcgcgtcc

SEQ ID NO: 16 - DNA, 45 bp, Artificial
  primer
ccgcgcctccgtcgacttggccggcagcaacagcttcagccaaac

SEQ ID NO: 17 - DNA, 45 bp, Artificial
  primer
gtttggctgaagctgtcgctgccgcccaagtcgacggaggcgcgg

SEQ ID NO: 18 - DNA, 45 bp, Artificial
  primer
cgcttcgccgtcgattacacgggtaacctttacagcatcggcgcg

SEQ ID NO: 19 - DNA, 45 bp, Artificial
  primer
cgcgccgatgctgtaaaggttacccgtgtaatcgacggcgaagcg

SEQ ID NO: 20 - DNA, 42 bp, Artificial
  primer
gcgcgggctgaagcctttgttacctgtttttgcgtgtgcggc

SEQ ID NO: 21 - DNA, 42 bp, Artificial
  primer
gccgcacacgcaaaaacaggtaacaaaggcttcagcccgcgc

SEQ ID NO: 22 - DNA, 45 bp, Artificial
  primer
ttgagcctcaaccgcgccgggggctccaacacctccatcggcctc

SEQ ID NO: 23 - DNA, 45 bp, Artificial
  primer
gaggccgatggaggtgttggagcccccggcgcggttgaggctcaa

SEQ ID NO: 24 - DNA, 45 bp, Artificial
  primer
ggacgcgccgatgctgtaaaggttacccgtgtaatcgacggcgaa

SEQ ID NO: 25 - DNA, 45 bp, Artificial
  primer
ttcgccgtcgattacacgggtaacctttacagcatcggcgcgtcc

SEQ ID NO: 26 - DNA, 15 bp, Artificial
  DNA modifications on modified nspA gene products
tggccggcagcaaca

SEQ ID NO: 27 - DNA, 10 bp, Artificial
  DNA modifications on modified nspA gene products
gggtaacctt

SEQ ID NO: 28 - DNA, 10 bp, Artificial
  DNA modifications on modified nspA gene products
taacaaaggc

SEQ ID NO: 29 - DNA, 10 bp, Artificial
  DNA modifications on modified nspA gene products
gggggctcca

SEQ ID NO: 30 - DNA, 10 bp, Artificial
  DNA modifications on modified nspA gene products
acgggtaacc

SEQ ID NO: 31 - protein, 15 aa, Artificial
  peptide
GSAKGFSPRISAGYR

SEQ ID NO: 32 - protein, 15 aa, Artificial
  peptide
VDLDAGYRYNYIGKV